(19) United States
(12) Patent Application Publication
(10) Pub. No.: US 2025/0065129 A1
(43) Pub. Date: Feb. 27, 2025

(54) CRANIAL NERVE STIMULATOR WITH RESPIRATION CYCLE DETECTION

(71) Applicant: AVIVOMED, INC., Roseville, MN (US)

(72) Inventors: Paul Richard Mazanec, Ham Lake, MN (US); Stephen C. Masson, Raleigh, NC (US)

(21) Appl. No.: 18/942,998

(22) Filed: Nov. 11, 2024

Related U.S. Application Data
(63) Continuation of application No. PCT/US2024/016383, filed on Feb. 19, 2024.
(60) Provisional application No. 63/446,930, filed on Feb. 20, 2023.

(51) Int. Cl. A61N 1/36

(52) U.S. Cl. CPC A61N 1/36139; A61N 1/3611

(57) ABSTRACT

An implantable neuromodulation system can include an electrode configured to be disposed at or near a first branch of a hypoglossal nerve of a patient and configured to provide a first electrostimulation therapy to treat a sleep disorder or breathing disorder of a patient. The system can include a sensor configured to provide a sensor signal that includes information about a patient response to the first electrostimulation therapy, information about a patient respiration, or information about one or more other physiologic status indicators for the patient. The sensor signal information can be used to quantify a progression of a breathing disorder for the patient.

### CLAIM OF PRIORITY

This application is a continuation of PCT/US2024/016383, filed on Feb. 19, 2024, and entitled “Cranial Nerve Stimulator with Respiration Cycle Detection,” which claims the benefit of U.S. Provisional Application No. 63/446,930, filed on Feb. 20, 2023, and entitled “Cranial Nerve Stimulator with Respiration Cycle Detection,” the entirety of which are incorporated herein by reference.

### BACKGROUND

Neural function can impact various disorders such as including cardiovascular disorders, movement disorders and tremors, epilepsy, depression, respiratory disorders (e.g., chronic obstructive pulmonary disease (COPD), pleural effusion), sleep disorders (e.g., obstructive sleep apnea (OSA)), obesity, xerostomia, and facial pain disorders. These disorders impact millions of patients and impact their quality of life and longevity. Obstructive sleep apnea, for example, is a common sleep disorder. Individuals suffering from OSA experience interrupted breathing patterns during sleep. Chronic, severe sleep apnea can require treatment to prevent sleep deprivation and other sleep-related complications. Obstructive sleep apnea is prevalent in patients with cardiovascular disease, is a cause of hypertension, and is associated with increased incidence of stroke, heart failure, atrial fibrillation, and coronary heart disease. Severe OSA is associated with an increase in all-cause and cardiovascular mortality.

In an example, external or implanted muscle stimulation devices or neurostimulation devices can be provided to excite tissue structures in or near an airway, such as to help treat sleep apnea or to counter apneic and hypopneic events.

In an example, neurostimulation can be used to treat a variety of disorders other than OSA. For example, neurostimulation can be used to treat epilepsy, depression, heart failure, obesity, pain, migraine headaches, COPD, or other disorders.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates generally a first anatomic example of front view of an anterior cervical region of a human.

FIG. 2 illustrates generally a second anatomic example that includes a portion of an anterior cervical triangle.

FIG. 3 illustrates generally a third anatomic example that includes a partial side view of an anterior cervical triangle.

FIG. 4 illustrates generally a fourth anatomic example that includes a partial side view of an anterior cervical triangle.

FIG. 5 illustrates generally a first implantable device implanted in a submandibular triangle of a patient.

FIG. 6 illustrates generally an example of a system that can be configured to provide a neuromodulation therapy.

FIG. 7 illustrates generally an example of an implantable system.

FIG. 8 illustrates generally an example of a portion of an implantable system that is configured to provide an electrostimulation therapy and sense a patient response to the neurostimulation therapy.

FIG. 9 illustrates generally an example of a physiologic parameter detection algorithm.

FIG. 10 illustrates generally an example of using respective time-divided portions of an accelerometer signal for different physiologic signal analyses.

FIG. 11 illustrates generally an example of a method for evaluating a quality of sleep for a patient.

FIG. 12 illustrates a routine 1200 for sensing physiologic status information using an implanted sensor in accordance with one embodiment.

FIG. 13 illustrates generally an example of a machine in the form of a computer system within which a set of instructions may be executed for causing the machine to perform any one or more of the methodologies discussed herein, according to an example embodiment.

### DETAILED DESCRIPTION

Systems, devices, and methods discussed herein can be configured for electrical stimulation of cranial nerves. Examples discussed herein can include methods for implanting a neuromodulation system or methods for using an implanted system to deliver neuromodulation therapy to one or more target cranial nerves, or to sense physiologic information about a patient, such as to monitor a disease state or control a neuromodulation therapy or other therapy. In an example, system or device features discussed herein can facilitate implantation of devices, leads, sensors, electrostimulation hardware, or other therapeutic means on or near cranial nerve tissue. In an example, the present subject matter includes systems and methods for implanting a neuromodulation device near or below an inferior border of a mandible (i.e., the body or ramus of the mandible or jaw bone) in an anterior triangle of the neck (e.g., located in the medial aspect), or in a posterior triangle of the neck (e.g., located in the lateral aspect), or in multiple regions of the neck.

The present inventors have recognized that a problem to be solved can include providing a minimally invasive neuromodulation therapy or treatment system that can provide signals to neural targets in or near a cervical region of a patient. The problem can include treating, among other things, obstructive sleep apnea (OSA), heart failure, hypertension, epilepsy, depression, post-traumatic stress disorder (PTSD), attention deficit hyperactivity disorder (ADHD), craniofacial pain syndrome, facial palsy, migraine headaches, xerostomia, atrial fibrillation, stroke, autism, inflammatory bowel disease, chronic inflammation, chronic pain, tinnitus, rheumatoid arthritis, or fibromyalgia. The problem can include providing an implantable system that can detect and respond to tongue movement or position to enhance an efficacy of an apnea treatment. The problem can further include providing an implantable system that can detect disordered breathing in coordination with, or concurrently with detecting other physiologic status information about a patient, such as patient posture, activity level, heart rate, or other characteristics. The problem can include providing such a system using a fewest number of different sensors and least power consumption.

The present inventors have recognized, among other things, that a solution to the above-described problems can include a neuromodulation system that can be implanted in an anterior cervical region of a patient, such as at or under a mandible of the patient. In an example, the system can include a housing that can be coupled to tissue in or near an anterior triangle, such as to digastric muscle or tendon tissue, to mylohyoid muscle tissue, to a hyoid bone, or to a mandible, among other locations. The present inventors have recognized that the solution can include or use a sensor, such as an accelerometer, implanted with the system and configured to sense information about tongue movement, motion, force, pressure, electrical activity, bioimpedance, or other information that can indicate tongue muscle behavior, upper airway air flow or breath (e.g., respiration), or a response to a stimulation therapy provided by the system. The present inventors have recognized that the same accelerometer can be used to sense information about patient respiratory cycle features, heart rate, posture, activity level, and more. The present inventors have further recognized that accelerometer signal processing can include or use time-multiplexing to extract or determine different information from respective different portions of the accelerometer signal.

The present inventors have recognized that the neuromodulation systems and methods discussed herein can be used to treat OSA, among other disorders or diseases. In an example, an OSA treatment can use a neuromodulation device that is implanted in one or more of a submental triangle and a submandibular triangle, and an electrode lead with electrodes that are configured to be disposed at or near one or more targets on a hypoglossal nerve, vagus nerve, glossopharyngeal nerve, or trigeminal nerve (e.g., at a mandibular branch of the trigeminal nerve). In an example, the solution can include using multiple electrodes or electrode leads to deliver a coordinated stimulation therapy to one or multiple cranial nerve targets. For example, the therapy can include bilateral stimulation of branches of the hypoglossal nerve, or stimulation of multiple different nerves. The therapy can be configured to selectively stimulate or block a neural pathway that influences activity of one or more of tongue muscles, mylohyoid muscles, stylohyoid muscles, digastric muscles, or stylopharyngeus muscles of a patient, to thereby treat OSA.

The description that follows describes systems, methods, techniques, instruction sequences, and computing machine program products that illustrate example embodiments of the present subject matter. In the following description, for purposes of explanation, numerous specific examples and aspects are set forth in order to provide an understanding of various embodiments of the present subject matter. It will be evident, however, to those skilled in the art, that embodiments of the present subject matter may be practiced in various combinations. Unless explicitly stated otherwise, structures (e.g., structural components, such as modules or functional blocks) are optional and may be combined or subdivided, and operations (e.g., in a procedure, algorithm, treatment, therapy, or other function) can vary in sequence or can be combined or divided.

In an example, the implantable neuromodulation systems and devices discussed herein can comprise a control system, signal or pulse generator, or other therapy signal generator, such as can be disposed in one or more housings that can be communicatively coupled to share power and/or data. The housings can comprise one or more hermetic enclosures to protect the circuitry or other components therein. In an example, a housing can include one or more headers, such as can comprise a rigid or flexible interface for connecting the housing, or circuitry or components inside of the housing, with leads or other devices or components outside of the housing. In an example, a header can be used to couple signal generator circuitry inside the housing with electrodes or sensors outside of the housing. In some examples, the header can house one or more sensors. In an example, the header can be used to couple circuitry inside the housing with a telemetry antenna, wireless power communication devices (e.g., coils configured for near-field communications or NFC), or other devices, such as can be contained within the header or disposed on or comprise flexible substrates or flexible circuits. This system configuration allows the housing(s), lead(s), and flexible circuits to be implanted in different anatomic locations, such as in a neck or cervical region of a patient. In an example, the various system components can be implanted in one or more of the anatomic triangular regions or spaces in the cervical region, and leads or other devices external to a circuitry housing can be tunneled to other locations, including at various cranial nerve targets. Accordingly, various therapeutic elements can be implanted on or near target cranial nerves, and sensing elements can be implanted on or near the same or other cranial nerves or at other anatomic structures in the same or different locations. Some components can be located in a different anatomic location, such as in a different cervical region than is occupied by a housing.

For example, a telemetry antenna or NFC coil can be provided at or near a surface of the skin, while a housing with circuitry that coordinates neuromodulation therapy or power signal management can be implanted elsewhere, such as more deeply within one of the anterior triangle spaces of the neck.

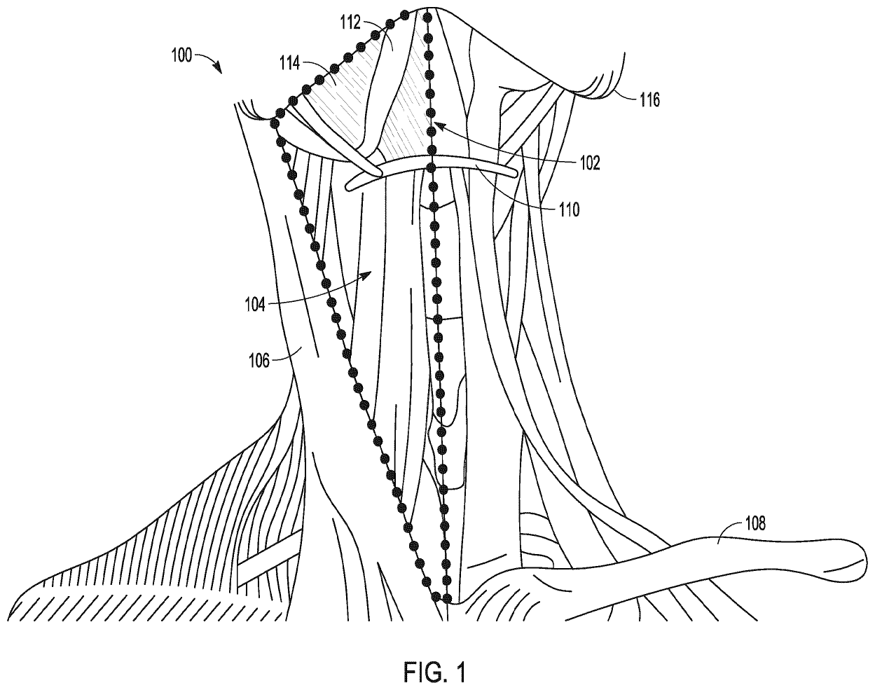

FIG. 1 illustrates generally a first anatomic example 100 of a front view of an anterior cervical region of a human. The region generally extends between a clavicle 108 and mandible 116 and can be divided into various additional regions or subregions. In an example, the anterior cervical region includes a pair of anterior triangles on opposite sides of a sagittal midline 102, such as including an anterior triangle 104 as illustrated. The term “midline” as used herein refers to a line or plane of bilateral symmetry in the cervical or neck region of a person. In an example, a midline corresponds to the sagittal plane, that is, is the anteroposterior (AP) plane of the body.

The anterior triangle 104 can include a region that is bounded by the midline 102, a base of the mandible 116, and a sternocleidomastoideole, or SCM 106. A hyoid bone 110 can extend between the pair of anterior triangles across the midline 102. The anterior triangle 104 can include, among other things, a digastric muscle 112 (e.g., including anterior and posterior portions of the digastric muscle 112), a mylohyoid muscle 114, and various other muscle, bone, nerve, and other body tissue.

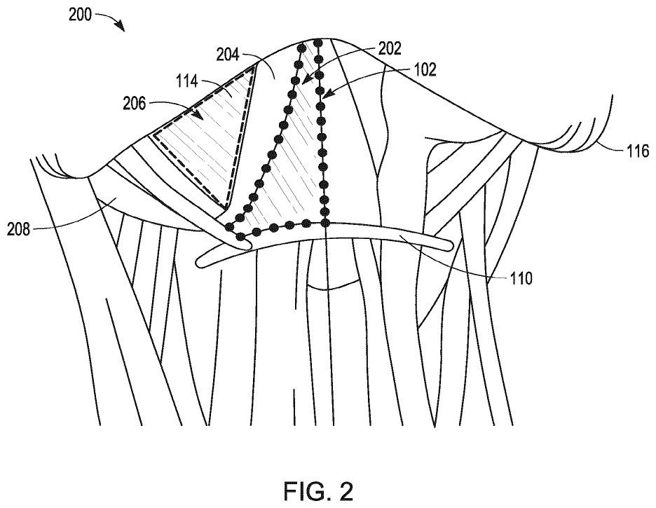

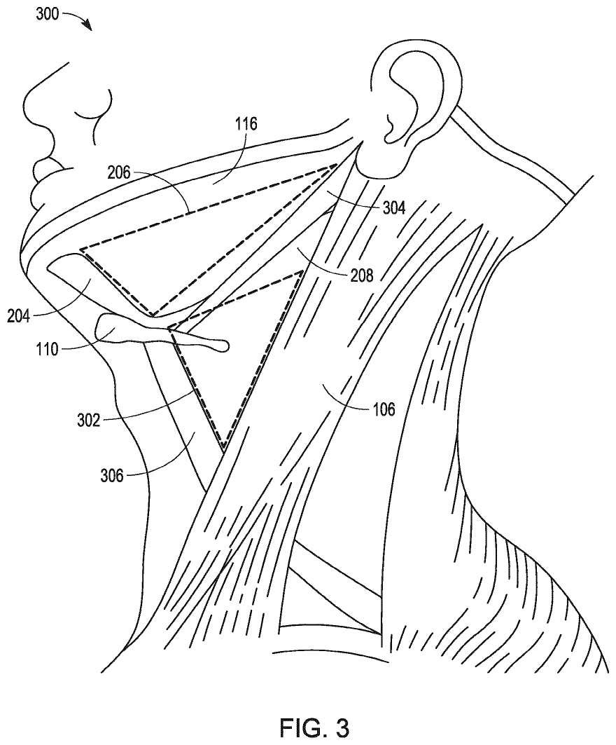

FIG. 2 illustrates generally a second anatomic example 200 that includes a portion of the anterior triangle 104 from the example of FIG. 1. FIG. 2 shows, for example, that the anterior triangle 104 can be divided into various regions, including a submandibular triangle 206, and a submental triangle 202. In an example, the anterior triangle 104 can further include a carotid triangle, as discussed below in the example of FIG. 3. A posterior triangle of the neck (not shown) can be divided into various regions, including an occipital triangle and a supraclavicular triangle.

The submental triangle 202 is generally understood to include a region that is bounded by the midline 102, the hyoid bone 110, and the anterior digastric muscle 204. The submandibular triangle 206 is generally understood to include a region that is bounded by the anterior digastric muscle 204, the posterior digastric muscle 208, and the base of the mandible 116.

FIG. 3 illustrates generally a third anatomic example 300 that includes a partial side view of the anterior triangle 104. The example of FIG. 3 further illustrates the location of the submandibular triangle 206, such as in relation to the anterior digastric muscle 204 and the mandible 116. The example of FIG. 3 illustrates the carotid triangle 302, such as can comprise a portion of the anterior triangle 104 in the cervical region. The carotid triangle 302 is generally understood to include a region that is bounded by the SCM 106, the omohyoid muscle 306, and the posterior digastric muscle 208.

In an example, an implantable neuromodulation device can be implanted in the anterior triangle 104 or in the posterior triangle, such as using the systems and methods discussed herein. In further examples, an implantable neuromodulation device can be implanted in one or more of the submental triangle 202 and the submandibular triangle 206. The implantable neuromodulation device can be configured to provide a stimulation therapy to one or multiple nerve targets such as can be in or near the anterior triangle 104 or the posterior triangle, or to nerve targets that can be accessed via tunneled leads that extend from a housing disposed in the anterior triangle 104 or the posterior triangle. In other words, various regions in the anterior and posterior cervical triangles can provide access to a main body of, or to branches of, various cranial nerves, including the hypoglossal nerve (CN XII), the accessory nerve (CN XI), the vagus nerve (CN X), the glossopharyngeal nerve (CN IX), the facial nerve (CN VII), and the trigeminal nerve (CN V), among others.

The present inventors have realized that the anterior and posterior cervical triangles are anatomic locations suitable for implantation of a neuromodulation system or component thereof. The present inventors have further realized that the locations include various anatomic structures suitable for coupling and therefore stabilizing a neuromodulation system or component thereof. For example, the present inventors have recognized that such coupling structures can include the hyoid bone 110, the connective tissue sling of the hyoid bone 110, the mandible 116, the digastric tendon, the anterior or posterior portion of the digastric muscle 112, the stylohyoid muscle 304, the mylohyoid muscle 114, the omohyoid muscle, or the SCM 106.

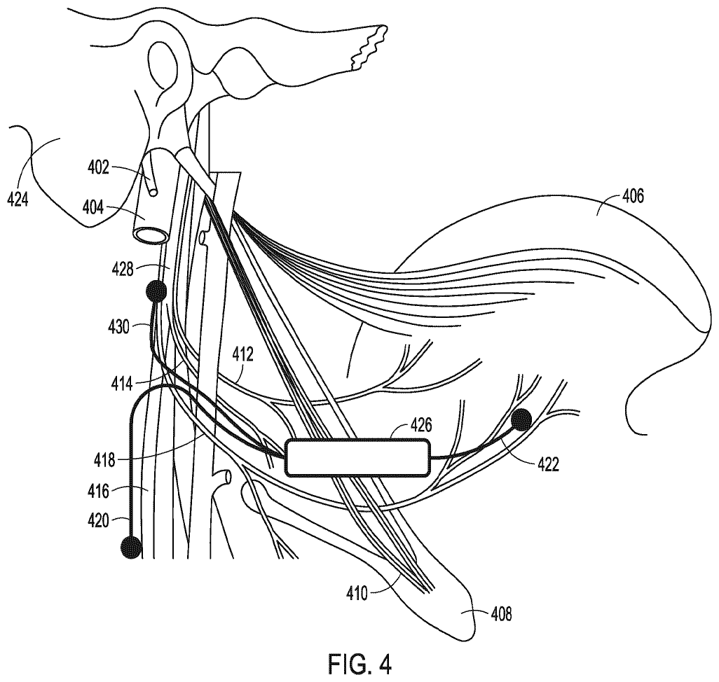

FIG. 4 illustrates generally a fourth anatomic example 400 that includes a partial side view that includes the anterior triangle 104. The fourth anatomic example 400 illustrates an upper portion of the anterior triangle 104 and a portion of the upper neck, such as at or below a temporal bone 424. A representation of a tongue 406 and of a portion of a jugular vein 404 is included for further context and reference.

The fourth anatomic example 400 shows various nerves and vessels. The illustrated nerves include some, but not all, of the cranial nerves that can be targeted using the neuromodulation systems, devices, and methods discussed herein. For example, nerve targets in the fourth anatomic example 400 include a facial nerve 402, a jugular vein 404, a glossopharyngeal nerve 412, a pharyngeal branch of vagus nerve 414, a vagus nerve 416, a hypoglossal nerve 418, and a mandibular branch of the trigeminal nerve 428, among others.

The example of FIG. 4 includes an example of an implantable therapy device 426. The implantable therapy device 426 can be implanted in a patient in an upper portion of an anterior triangle 104 of a cervical region of the patient. For example, the implantable therapy device 426 can be implanted in one or more of the submental triangle 202 and the submandibular triangle 206. In the example of FIG. 4, the implantable therapy device 426 can be coupled to various anatomical structures, such as a stylohyoid muscle 410, a hyoid bone 408, or other tendons or structures in the upper neck.

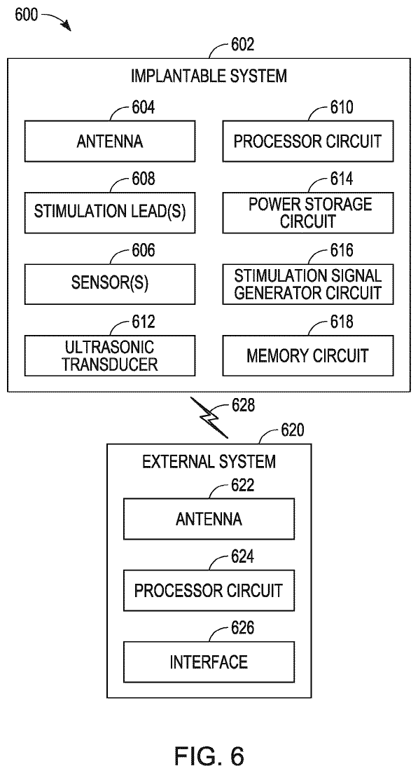

The example of FIG. 4 includes multiple leads coupled to the implantable therapy device 426. For example, the implantable therapy device 426 can be coupled to a lower electrode lead 420, an anterior electrode lead 422, and an upper electrode lead 430. The lower electrode lead 420 can be implanted at or near a neural target on the vagus nerve 416, for example, in or adjacent to the carotid triangle 302. In an example, the lower electrode lead 420 can be coupled to the SCM 106 or other structure at or near the vagus nerve 416. The upper electrode lead 430 can be implanted at or near the facial nerve 402, the mandibular branch of the trigeminal nerve 428, or the glossopharyngeal nerve 412, among others. In an example, the anterior electrode lead 422 can be implanted at or near a neural target on the hypoglossal nerve 418. Various details of the implantable therapy device 426 and its associated leads are discussed herein, including in the example of FIG. 6.

In an example, the various implantable devices and components thereof that are discussed herein can be coupled to various anatomic structures or tissues inside a patient body, such to stabilize or maintain a device or component at a particular location and resist device movement or migration as the patient carries out their daily activities. In an example, coupling a device or component to tissue can include anchoring, affixing, attaching, or otherwise securing the device or component to tissue using a coupling feature. A coupling feature can include, but is not limited to, a flap or flange, such as for suturing to tissue (e.g., muscle, tendon, cartilage, bone, or other tissue).

In an example, a coupling feature can include various hardware such as a screw or helical member that can be driven into or attached to tissue or bone. In an example, a coupling feature can include a cuff, sleeve, adhesive, or other component. In an example, one or multiple different coupling features can be used for different portions of the same neuromodulation system. For example, a suture can be used to couple a device housing to a tissue site, and a lead, such as coupled to the housing, can include a distal cuff to secure the lead at or near a neural target.

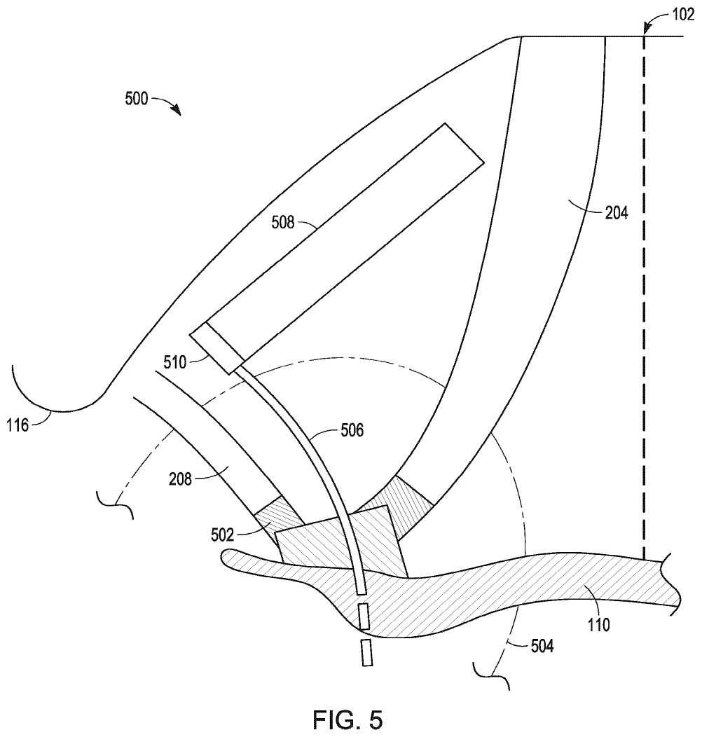

FIG. 5 illustrates generally a first example 500 that includes a first implantable device 508 implanted in the submandibular triangle 206 of a patient. In the first example 500, the first implantable device 508 can be coupled to an anatomic structure in the submandibular triangle 206, such as using a suture, anchor, or other affixation means. In an example, the first implantable device 508 can be coupled to one or more of the mandible 116, the anterior digastric muscle 204, the posterior digastric muscle 208, the mylohyoid muscle 114, the digastric tendon 502, or other bone, tendon, muscle, or other structure that is in or adjacent to the submandibular triangle 206. In the example of FIG. 5, the first implantable device 508 can be provided near, but spaced apart from, a submandibular gland 504 of the patient.

In the example of FIG. 5, the first implantable device 508 includes a first header 510. The first header 510 can be used to couple one or multiple electrode leads, sensor leads, or other devices to the first implantable device 508. For example, the first header 510 can be used to couple the first implantable device 508 to a first electrode lead 506, and the first electrode lead 506 can be tunneled to a cranial nerve target. Electrodes configured to deliver electrostimulation signals to the nerve target can be situated at or adjacent to the target. In an example, the first electrode lead 506 can be tunneled to a hypoglossal nerve in or near an anterior cervical region of a patient.

In the example of FIG. 5, the first implantable device 508 is shown with one header. The first implantable device 508 can optionally include multiple headers to interface the first implantable device 508 with one or multiple other leads, such as electrode leads, sensor leads, communication coils, or other devices. Referring again to FIG. 3, for example, the implantable therapy device 426 can include multiple headers, such as coupled to the respective different leads that extend from opposite sides of a body of the implantable therapy device 426.

FIG. 6 illustrates generally an example of a system 600 that can be configured to provide or control a neuromodulation therapy. The system 600 can include an implantable system 602 and an external system 620. The implantable system 602 and the external system 620 can be communicatively coupled using a wireless coupling 628. In an example, the wireless coupling 628 can enable power signal communication (e.g., unidirectionally from the external system 620 to the implantable system 602), or can enable data signal communication (e.g., bidirectionally between the implantable system 602 and the external system 620). In an example, the implantable system 602 or the external system 620 can be wirelessly coupled for power or data communications with one or more other devices, including other implantable or implanted devices, such as in the same patient body.

In the example of FIG. 6, the implantable system 602 can include an antenna 604, a sensor(s) 606 such as comprising one or more physiologic sensors, a stimulation lead(s) 608, a processor circuit 610, an ultrasonic transducer 612, a power storage circuit 614, a stimulation signal generator circuit 616, and a memory circuit 618, among other components or modules.

In an example, the antenna 604 can include a telemetry antenna such as configured for data communication between the implantable system 602 and the external system 620. In an example, the antenna 604 can include an antenna, such as an NFC coil, that is configured for wireless power communication between the implantable system 602 and the external system 620 or other external power source.

The processor circuit 610 can include a general purpose or purpose-built processor. The memory circuit 618 can include a long-term or short-term memory circuit, such as can include instructions executable by the processor circuit 610 to carry out therapy or physiologic monitoring activities for the system 600. In an example, the processor circuit 610 of the implantable system 602 is configured to manage telemetry or data signal communications with the external system 620, such as using the antenna 604 or other communication circuitry.

In an example, the stimulation signal generator circuit 616 includes an oscillator, pulse generator, or other circuitry configured to generate electrical signals that can provide electrostimulation signals to a patient body, or to power various sensors (e.g., including the sensor(s) 606), or transducers (e.g., including the ultrasonic transducer 612). In an example, the stimulation signal generator circuit 616 can be configured to generate multiple electrical signals to provide multipolar electrostimulation therapy to multiple neural targets, such as concurrently or in a time-multiplexed manner. The stimulation signal generator circuit 616 can be configured to use or provide different neurostimulation signals, such as can have different pulse amplitude, pulse duration, waveform, stimulation frequency, or burst pattern characteristics.

The stimulation signal generator circuit 616 can be used to generate therapy signals for multiple different targets concurrently. For example, signals from the stimulation signal generator circuit 616 can be used to stimulate one cranial nerve target to efferent effect, and to stimulate a different nerve or branch to elicit an afferent response. In another example, one cranial nerve can be blocked while another nerve is stimulated. Other combinations can similarly be used.

In an example, the stimulation lead(s) 608 can include one or more leads that are coupled to or integrated with a housing or header of the implantable system 602. The stimulation lead(s) 608 can be detachable from the housing to facilitate replacement or repair.

In an example, the stimulation lead(s) 608 can include electrostimulation hardware such as electrodes having various configurations, including cuff electrodes, flat electrodes, percutaneous electrodes or other configurations suitable for electrical stimulation of nerves or nerve bodies or branches. In an example, the stimulation lead(s) 608 can additionally or alternatively comprise other neuromodulation therapy hardware such as the ultrasonic transducer 612, drug delivery means, or a mechanical actuator, such as can be configured to modulate neural activity. The stimulation lead(s) 608 can include one or more electrodes that are configured to sense electrical activity from a patient body. For example, one or more of the electrodes can be configured to monitor an electrical response from nerve or muscle tissue of the patient body. In an example, the one or more electrodes of the stimulation lead(s) 608 can be used to receive information about an evoked compound action potential, or ECAP, such as can indicate a type or amount of neural fiber that is activated in response to a stimulation. In some examples, the stimulation can be provided using one or more of the same electrodes in the stimulation lead(s) 608 as used to receive the ECAP information, or the stimulation can be provided using other electrodes. The processor circuit 610 can be configured to receive the information about the ECAP and identify characteristics of the evoked response, such as can be used to assess an effectiveness of a neuromodulation therapy.

The leads and/or electrodes discussed herein can have various features that can facilitate placement at, and stimulation of, one or more neural targets. A lead can have one or more electrodes that can be used for nerve stimulation, nerve blocking, or nerve sensing. The electrodes can have various surface area and spacing (e.g., spacing from other electrodes, sensors, targets, etc.) to optimize for a particular function. In an example, an electrode can comprise various materials, including low-oxidation metals or metal alloys (e.g., platinum, platinum iridium, etc.) for use in implantable systems. In an example, an electrode can be treated or coated with another material such as to promote healing or enhance charge transfer to tissue.

In an example, an electrode lead can comprise one or multiple electrodes, such as can have the same or different electrode characteristics. A lead can include, for example, a spiral electrode or cuff electrode. In such an example, one or more conductive surfaces can be exposed on an inside surface of a curved or spiral cuff assembly such as can comprise a portion of a lead body. In an example, a spiral cuff assembly (and hence, electrodes) can be designed to circumferentially wrap snugly around a body of a nerve and can be self-sizing. In an example, a cuff electrode can be configured to surround a particular target to thereby direct stimulation energy to the target from multiple different directions concurrently, such as while insulating the electrode from adjacent tissue.

In an example, a surface electrode or electrode array can be used. In this example, one or more electrodes can be exposed on one side of a flat or round section of a lead body. An array of electrodes of various shapes, sizes, or other characteristics, can be provided to spatially control neuromodulation therapy delivery. In an example, electrode surfaces can be oriented toward a target nerve or other structure, such as to focus an electric field provided by the electrode or electrodes. Surface electrode leads can be surgically placed by exposing the target anatomy, or can be steered using, e.g., a catheter-based delivery system from a distal surgical access point.

In an example, a percutaneous electrode can be used, such as including one or more electrodes exposed on a lead that is inserted into a blood vessel (or other conducting tissue in the vicinity of a neural target) using percutaneous techniques. A percutaneous lead can be navigated by a clinician, within or through vasculature, toward target nerves or neural structures that are in close proximity to the vasculature. In an example, electrodes on a percutaneous lead can be directly on the lead body or can comprise a percutaneous structure, such as a stent-like frame or scaffold, whereby the electrodes can be oriented towards the target and away from the blood in the vessel.

In an example, a bifurcated lead can be used to provide electrodes at multiple different and spaced apart anatomical targets while using a single connection to a header. In an example, a modular lead can be used such as to extend or tailor a lead to accommodate a patient's anatomy or target structures.

In an example, the stimulation lead(s) 608 can comprise one or more electrodes that can be provided or grouped together at a distal end of a lead, such as spaced apart from a housing, or the electrodes can be distributed along a length of the lead. In an example, a lead can include multiple different electrode groups of one or more electrodes provided at different locations along a length of the lead. Additionally, a housing of the various devices discussed herein can include one or more electrodes configured for use in electrostimulation delivery. Each of the electrodes in or coupled to the implantable system 602 can be separately addressable by neuromodulation therapy control or coordination circuitry to deliver a coordinated therapy to one or multiple targets, or to sense a response (e.g., an ECAP response) at one or multiple neural tissue areas.

Various stimulation configurations can be used with any of the electrode or lead types discussed herein. In an example, different configurations can be used to provide or modify a stimulating electric field to thereby affect an extent and manner of neural excitation. The configurations can include, for example, unipolar, bipolar, and various combinations of multipolar configurations. In a bipolar or multipolar configuration, a guard electrode can be used to help steer excitation or inhibit neural activity. In an example, an electrode configuration can be dynamically changed, such as throughout the course of a particular therapy, such as through programming changes or during operation to achieve a particular therapy.

In an example, the sensor(s) 606 can include, among other things, electrodes for sensing of electrical activity such as using electrocardiograms (ECGs), impedance, electromyograms (EMGs) of select muscles, and/or electroneurograms (ENGs) of target cranial nerves and branches. The sensor(s) 606 can include pressure sensors, photoplethysmography (PPG) sensors, chemical sensors (e.g., pH, lactate, glucose, etc.) or other sensors that can be used for physiologic sensing of cardiac, respiratory, or other physiologic activity. In an example, the sensor(s) 606 can include an accelerometer, gyroscope or geomagnetic sensor, such as can be configured to measure patient or device movement, vibration, position, posture, or other orientation information. Other examples of the sensor(s) 606 are discussed elsewhere herein, including in the discussion of the machine 1300 and the various I/O components 1342, such as including the biometric components 1332, motion components 1334, and environmental components 1336. In an example, information from the sensor(s) 606 can be received by the processor circuit 610 and used to update or titrate a neuromodulation therapy.

In an example, the implantable system 602 can include one or more sensor(s) 606, such as can be used in providing closed-loop neuromodulation therapy that is based at least in part on physiologic status information about a patient (e.g., respiration, heart rate, blood pressure, neural or muscular activation, or other information). In an example, the sensor(s) 606 can be used to receive diagnostic information, or to receive information about patient movement or body position or posture. Data from the sensor(s) 606 can be sampled at various different rates, or data can be sampled from a single sensor at multiple different rates, such as depending upon the type of physiologic information to be derived from the data.

In an example, hypoglossal nerve stimulation, such as to treat OSA, can be controlled at least in part based on information from an accelerometer or gyroscope to determine patient respiration, patient activity, and body orientation or position, such as together with information from a pressure sensor about respiration. In other words, using information from the sensor(s) 606, such as including accelerometer and pressure sensors, the implantable system 602 can control neuromodulation therapy provided to the hypoglossal nerve, such as can include stimulation during a particular time within a respiratory cycle, and can use body position information to automatically enable therapy when, for example, the patient is sleeping. In some examples, information from the sensor(s) 606 can be used to determine a sleep stage, and information about the sleep stage can be used to titrate therapy.

In an example, timing characteristics of an accelerometer signal can be analyzed through a series of signal processing steps to extract information regarding the patient's respiration, posture, activity level, heart rate, or other characteristic information about the patient's health status. For example, in respiration analysis, the accelerometer signal can be processed to isolate low- frequency components that correspond to the patient's breathing cycle. In an example, the components include low-frequency (e.g., about 1 Hz or less) information about respiration-related motion, or include higher-frequency (e.g., about 30-60 Hz) acoustic or other soft tissue-related information. In an example, the analysis includes applying a filter to raw accelerometer data to attenuate frequencies outside of a desired frequency range. Envelope detection of the filtered signal can be used to demodulate the filtered signal and extract an amplitude modulation envelope that corresponds to the expansion and/or contraction of the thoracic cavity during breathing. In an example, the accelerometer signal can be sampled at different rates depending on the particular components to be analyzed. For example, a lower sampling rate can be used for the accelerometer signal when the system is configured to determine the respiration-related information from motion, and a higher sampling rate can be used when the system is configured to determine the respiration-related information from acoustic information.

Posture and activity level determination can be based on static and dynamic components of the accelerometer signal, respectively. The static component, which can indicate the gravitational force vector, provides information about the patient's orientation relative to the ground. By analyzing the changes in this component over time, the system can infer the patient's posture, such as lying down, sitting, or standing. The dynamic component, on the other hand, reflects patient movement and can be used to determine patient activity levels. By monitoring the frequency and intensity of movements detected by the accelerometer, the system can distinguish between states of rest and periods of activity. In some examples, the periods of activity can be further analyzed to distinguish periods of rest or sleep from other daily activities, or to distinguish restful sleep from non-restful sleep.

Heart rate information can be determined from the accelerometer signal by identifying the subtle tissue oscillations associated with the mechanical activity of the heart. The accelerometer signal can be processed to filter out motion artifacts and isolate the frequency band corresponding to the expected heart rate range. Signal processing techniques, such as autocorrelation or spectral analysis, can be applied to the filtered signal to detect the periodicity of the heartbeats and calculate the heart rate.

In an example, information from multiple different sensors can be used together to cross-check or validate physiologic status information, or to help improve immunity from noise or other aberrations in sensor data. In some examples, primary and secondary sensors can be used together, and information from the secondary sensor can be used in the event of a primary sensor failure or unavailability. In an example, different types of information received or derived from the same sensor can be used together to cross-check or validate physiologic status information, or to help improve immunity from noise or other aberrations in sensor data. For example, respiration information and heart rate information from the same accelerometer signal can be used together to determine or validate a patient health status. In an example, respective different types of data can be sampled from the same sensor at respective different rates.

In the context of respiration cycle determination, the accelerometer signal can be analyzed to detect movement-related information indicative of the patient's breathing. Lower-frequency components of the accelerometer signal, typically at about 1 Hz or less, are associated with the physical motion of the thoracic cavity during respiration. The system is configured to process this lower-frequency information to determine the respiration cycle, including the rate and regularity of breathing.

To enhance accuracy of respiration cycle determination, the system can use higher-frequency respiration-related information, such as within the range of about 30-60 Hz. This higher-frequency information can be derived from the accelerometer signal and can indicate acoustic vibrations caused by airflow during respiration. The system is configured to use this higher-frequency information to validate the respiratory cycle information determined using the lower-frequency information. In other examples, the system can be configured to use the lower-frequency motion-related information to validate respiration cycle information determined using the higher-frequency information.

Analysis of the higher-frequency information can be selectively used, particularly when there is a need to confirm suspected respiratory events such as apnea. In an example, the system dynamically updates the sampling rate of the accelerometer signal to capture the higher-frequency information with greater precision. This adaptive approach to signal analysis allows for efficient use of the system's resources, conserving power by increasing the sampling rate only when higher-resolution data is required for event confirmation.

In an example, the processor circuit 610 or the external system 620 can be configured to use information about a therapy to receive or interpret data from the sensor(s) 606. For example, some sensor information may be corrupted or otherwise influenced by an electrostimulation therapy provided by the implantable system 602 or by another therapy or event provided by the same or other implanted or external device. In some examples, sensor information can be “blanked” or disregarded at or during a stimulation therapy delivery event.

In the example of FIG. 6, the external system 620 can include various components that can be provided together as a unitary external device or can include multiple devices configured to work together to manage a patient therapy, manage a device such as the implantable system 602, or perform other functions associated with the implantable system 602. The external system 620 can include an antenna 622, a processor circuit 624, and an interface 626, among other components or modules.

The antenna 622 can comprise one or multiple antennas such as can be configured for nearfield or farfield communications with, for example, the antenna 604 of the implantable system 602, a different implantable device or system, or other external device. In an example, the antenna 622 and the antenna 604 can be used to exchange power or data between the implantable system 602 and the external system 620. For example, information about a prescribed therapy can be uploaded from the external system 620 to the implantable system 602, or information about a physiologic status, such as measured by the sensor(s) 606, can be downloaded from the implantable system 602 to the external system 620.

The processor circuit 624 can include a general purpose or purpose-built processor configured to carry out various activities on the external system 620 or in coordination with the implantable system 602. In an example, the processor circuit 624 of the external system 620 is configured to manage telemetry or data signal communications with the implantable system 602, such as using the antenna 622 or other communication circuitry.

The interface 626 can include a patient or clinician interface, such as to report device information or to receive instructions or therapy parameters for implementation by the implantable system 602. In an example, the interface 626 can include an interface or gateway to facilitate communication between the 602 or the external system 620 with a patient management system or other medical record system. Other features, modules, and components of the implantable system 602 and the external system 620 can be included in the system 600 to help administer various neuromodulation therapies.

In an example, the systems, devices, and components discussed herein, including at least the implantable system 602 and the external system 620 of the system 600, can be used to provide neuromodulation therapy to nerve targets inside a patient body, such as to treat one or more disorders or diseases. In an example, the system 600 or components thereof can be configured to provide neuromodulation therapy to multiple nerve targets in a coordinated manner, such as concurrently, or in a time-multiplexed sequence. In an example, the neuromodulation therapy can include one or more, or combinations of, neural stimulation and blocking signals, such as can be directed to afferent or efferent nerve structures or targets to trigger different responses. The therapy can optionally include using vector-based stimulation configurations to target particular nerves or nerve regions, or can include more relatively targeted or isolated nerve fibers. In an example, a coordinated neuromodulation therapy can include blocking at a first nerve target, while stimulating a second nerve target, or concurrently (or in time-sequence) stimulating multiple different nerve targets.

In an example, the particular patient disorder or disease can dictate the particular neural target to modulate with a neuromodulation therapy. For example, to treat obstructive sleep apnea using the system 600, various cranial nerves can be targeted individually or together, such as including the trigeminal nerve (e.g., the V3 mandibular branch of the trigeminal nerve 428), the hypoglossal nerve 418 (e.g., including one or more branches thereof), the glossopharyngeal nerve 412, the vagus nerve 416, or the facial nerve 402 (eg., including various extracranial branches thereof).

In an example, the system 600 can be used to treat OSA by providing a neuromodulation therapy to or including the mandibular branch of the trigeminal nerve 428 and the hypoglossal nerve 418. In this example, neuromodulation of the mandibular branch of the trigeminal nerve 428 can influence motor control of the mylohyoid muscle 114 or the anterior digastric muscle 204, and neuromodulation of the hypoglossal nerve 418 can influence motor control of muscles in the tongue 406.

In an example, the system 600 can be used to treat OSA by providing a neuromodulation therapy to or including the facial nerve 402 and to the hypoglossal nerve 418. In this example, neuromodulation of the facial nerve 402 can influence motor control of the stylohyoid muscle 304 or the posterior digastric muscle 208, and neuromodulation of the hypoglossal nerve 418 can influence motor control of muscles in the tongue 406.

In an example, the system 600 can be used to treat OSA by providing a neuromodulation therapy to or including the glossopharyngeal nerve 412 and the hypoglossal nerve 418. In this example, neuromodulation of the glossopharyngeal nerve 412 can influence motor control of the stylophryngeus muscle, and neuromodulation of the hypoglossal nerve 418 can influence motor control of muscles in the tongue 406.

In an example, the system 600 can be used to treat OSA by providing a neuromodulation therapy to or including various branches of the hypoglossal nerve 418, including anterior branches, posterior branches, or multiple branches concurrently, including or using a bilateral configuration to target branches on opposite sides of the midline 102 of a patient. The neuromodulation of the hypoglossal nerve 418 can influence motor control of various muscles in the tongue 406. In an example, neuromodulation therapy that includes stimulating or blocking the hypoglossal nerve 418 can be combined with therapy that targets one or more of the mandibular branch of the trigeminal nerve 428 (e.g., to influence motor control of the mylohyoid muscle 114 or the anterior digastric muscle 204), the facial nerve 402 (e.g., to influence motor control of the stylohyoid muscle 304 or the posterior digastric muscle 208), or the glossopharyngeal nerve 412 (e.g., to influence motor control of the stylophryngeus muscle), among others.

Any one or more branches of the hypoglossal nerve 418 can receive a neuromodulation therapy from the implantable system 602. For example, any one or more of the posterior branches of the hypoglossal nerve 418 can receive neuromodulation, including for example “branches” off the hypoglossal nerve sheath such as the meningeal branch (B1), the vascular branch (B2), the descending branch, also referred to as the superior root of the ansa cervacalis (B3), the thyrohyoid branch (B4), or the geniohyoid branch (B5). Any one or more of the anterior branches of the hypoglossal nerve 418 can receive neuromodulation, including for example where a main trunk of the hypoglossal nerve 418 branches to the muscles of the tongue, also referred to as the muscular branch (B6), or including the muscular branch itself. The muscular branch can include sub-branches or nerve fibers that innervate specific muscles of the tongue.

In an example, the system 600 can be used to treat OSA or other disorders or diseases such as heart failure, hypertension, atrial fibrillation, epilepsy, depression, stroke, autism, inflammatory bowel disease, chronic inflammation, chronic pain (e.g., in cervical regions, in the lower back, or elsewhere), tinnitus, or rheumatoid arthritis, among others, such as by providing a neuromodulation therapy to or including the vagus nerve 416. Neuromodulation of the vagus nerve 416 can influence parasympathetic tone to thereby treat or alleviate symptoms associated with the various diseases or disorders mentioned, among others. In an example, a therapy that includes stimulation of the vagus nerve 416 can include therapy provided to one or more branches of the hypoglossal nerve 418, the mandibular branch of the trigeminal nerve 428, the facial nerve 402, or the glossopharyngeal nerve 412. In an example, neuromodulation therapy that includes stimulating or blocking a portion of the vagus nerve 416 can be combined with therapy that targets one or more of the glossopharyngeal nerve 412 (e.g., to further influence parasympathetic tone), the carotid sinus (e.g., to stimulate a baroreceptor response), or the superior cervical ganglion or branches thereof (e.g., to influence sympathetic tone).

In an example, a neuromodulation therapy for treatment of heart failure, hypertension, and/or atrial fibrillation can include therapy provided to or including one or more of the glossopharyngeal nerve 412 (e.g., to influence parasympathetic tone, such as via communication to the vagus nerve 416), the superior cervical ganglion (e.g., to influence sympathetic tone), or the carotid sinus (e.g., to stimulate a baroreceptor response).

In an example, the system 600 can be configured to treat heart failure, hypertension, migraine headaches, xerostomia, or other diseases or disorders by providing a neuromodulation therapy to or including the glossopharyngeal nerve 412. Stimulation or blocking of the glossopharyngeal nerve 412 can, for example, influence parasympathetic tone or can affect motor activity of the stylopharyngeus muscle.

In an example, the system 600 can be configured to treat drug-refractory epilepsy, depression, post-traumatic stress disorder (PTSD), migraine headaches, attention-deficit hyperactivity disorder (ADHD), craniofacial pain syndrome, among other diseases and disorders, such as by providing a neuromodulation therapy to or including the mandibular branch of the trigeminal nerve 428.

In an example, the system 600 can be configured to treat craniofacial pain syndrome, or facial palsy, among other things, such as by providing a neuromodulation therapy to or including the facial nerve 402, such as including various extracranial branches or roots thereof. In an example, the system 600 can be configured to treat fibromyalgia such as by providing a neuromodulation therapy to or including the spinal accessory nerve, such as to target the trapezius muscle, which is understood to be a potential trigger point for fibromyalgia. In an example, the system 600 can be configured to treat migraine headaches or tinnitus, such as by providing a neuromodulation therapy to or including a great occipital nerve, such as can be accessed using electrodes implanted in the cervical region of a patient.

Neuromodulation therapies can thus be provided using the system 600, or using components thereof, to treat a variety of different diseases or disorders. The therapies can include targeted, single-location stimulation or blocking (e.g., using electrical pulses, ultrasonic signals, or other energy) therapy at one of the locations mentioned herein (among others) or can include coordinated stimulation or blocking across or using multiple different locations. The following discussion illustrates several examples of different implantation locations and neural targets, however, others including those specifically mentioned above, can similarly be used.

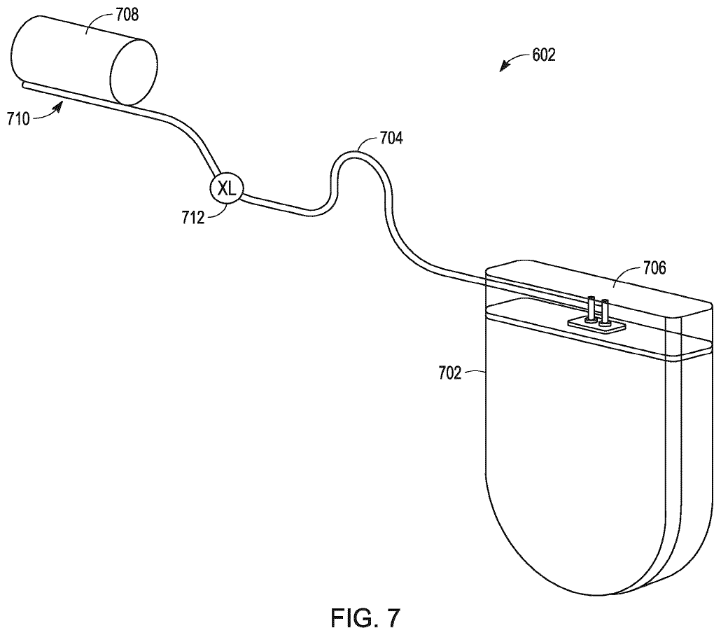

In an example, the implantable system 602 comprises an implantable housing (sometimes referred to as a “can”) or body portion that includes a flattened or compressed half-capsule (or other portion of a capsule) structure, as shown in FIG. 7. The housing can extend along a longitudinal axis and includes rounded ends or caps. In an example, the housing is a conductive housing 702 that can be configured as a reference electrode for use in electrostimulation delivery or electrical response sensing.

The implantable body can include a header 706 for interfacing with one or multiple leads. In the example of FIG. 7, the implantable body can be coupled to a single lead 704 having a distal cuff 708 that includes an electrode array 710. The cuff 708 can be configured for placement on or around a medial branch of the hypoglossal nerve or other neural tissue. The lead 704 can optionally include a suture anchor at or near the distal end, at or near the proximal (header) end, or in a central portion thereof.

In an example, the implantable system 602 comprises a cranial nerve stimulator with one or more housings and one or more stimulation leads, and is configured to be implanted in an anterior cervical region, at or near one or more cranial nerves. One or more of the sensor(s) 606 in the implantable system 602 can be configured to sense physiologic signals, sometimes referred to herein as feedback signals. Such physiologic signals, or information therein, can indicate a therapeutic or diagnostic effect. The sensor(s) 606 can be provided inside or outside of the stimulator housing(s) or can be provided on the lead 704. Some examples of the sensor(s) 606 include one or more of an accelerometer 712, motion sensor, acoustic transducer, pressure sensor, optical sensor, photoplethysmography sensor, chemical sensor, electrodes (e.g., on the stimulation lead(s) 608) to sense an ECAP signal or other electrical activity of neural or muscular structures, or one or more other sensors to measure a therapeutic or diagnostic effect.

In an example, a physiologic response that indicates a therapeutic or diagnostic effect can be a function of, or indicated by, one or more of motion, sound, head or neck posture, activity level, force, pressure, vascular changes, pleural cavity changes, electrical activity, bioimpedance change or other information. For example, characteristics of the sensed response can be determined from sensor signal characteristics, for example, using sensor signal information from the time domain (such as a signal amplitude, duration, rise or fall time, slope, period, integral, differential, or other timing characteristic) or from the frequency domain (e.g., signal spectral content).

In an example, physiologic feedback can comprise information about a change in a sensor signal. The feedback can be classified or categorized based on therapeutic effect or diagnostic value. In some examples, therapeutic effect or diagnostic value can be sensed by sensors dedicated to these functions or by sensors that also sense other physiologic information. Using the sensor information or feedback, the nerve stimulator system (e.g., the system 600 from the example of FIG. 6, or a portion thereof) can be used to modulate or titrate therapy automatically or can be used to communicate the sensor information to a clinician via remote or clinic-based follow-up. Subsequently, the clinician can update or titrate the therapy via programmable parameter changes.

In an example, the implantable system 602 of the system 600 includes a hypoglossal nerve (HGN) stimulator configured to treat obstructive sleep apnea. The implantable system 602 can be implanted in one or more of a submental and or submandibular triangle of the neck. The implantable system 602 can include or use one or more stimulation leads placed on the hypoglossal nerve(s) of a patient, and can be configured to use electrostimulation therapy to control an upper airway patency-related muscle, for example, the tongue. For example, the implantable system 602 can include one or more electrodes configured to deliver a therapy to induce a change a position of, or to otherwise influence movement of, the tongue, such as to relieve an upper airway obstruction. In some examples, following implantation of the implantable system 602, a clinician adjusts or titrates neurostimulation parameters to achieve a desired tongue movement, such as an excursion of the tongue away from the airway.

Tongue movement, such as in response to an electrostimulation, can manifest as motion, force, pressure, electrical activity (such as an electromyogram signature), bioimpedance change or other effect in various regions of the neck. Furthermore, any resulting upper airway change or obstruction relief can manifest as a change in an acoustic, pressure, or bioimpedance change that can be detected in or near the neck.

In an example, the sensor(s) 606 and/or the processor circuit 610 can be configured to sense or determine diagnostic information such as can include information about a number of apnea or hypopnea events, absence, presence or other characteristics of snoring or other vocalizations, and a general condition of the patient (such as can be indicated by posture or activity level, such as relative to a patient-specific or population-specific reference or baseline). Other information from the sensor(s) 606 and/or the processor circuit 610 can be used to measure tongue movement effects, detect a status of the upper airway, or to use the sensed information to develop a desired or target therapy signature or pattern. The implantable system 602 can then be configured to modulate or titrate therapy based on the desired signature or pattern. Alternatively or additionally, the implantable system 602 can be configured to store target therapy signature or pattern information and provide such information via remote or in-clinic follow-up so that a clinician can update the therapy. In an example, historical sensor information can be used to create other signatures or patterns, such as can be used with more recent sensor information to help predict future physiologic events like inspiration timing or breathing interruption.

In an example, the implantable system 602 can include an accelerometer or other sensor configured to measure tongue movement and position. The accelerometer or other sensor can be configured to concurrently sense tracheal sounds or vibrations from the patient's upper airway, such as to monitor for a presence of apnea or hypopnea events, or to detect snoring or other acoustic information. The accelerometer or other sensor can further be configured to detect a head, neck, or other body part orientation or posture, or to detect a sleep state. Using information about any one or more of these attributes, the HGN stimulator can develop a signature of a desired physiologic response in a variety of conditions to determine a proper therapy or to determine set of stimulation parameters to be applied to achieve a particular therapy result.

In an example, the HGN stimulator can monitor an accelerometer signal concurrently with, or following, delivery of a stimulation signal, to thereby observe tongue motion or other physiologic response information. In an example, a desired tongue motion can be identified, at least in part, by a rapidly rising or changing acceleration signal, as detected by an accelerometer positioned in the head or neck. In an example, less tongue movement can correspond to a diminished accelerometer signal amplitude, while more tongue movement can correspond to a relatively greater signal amplitude. A reference signal amplitude can be associated with a target or desired tongue movement. The HGN stimulator can be configured to automatically adjust a stimulation characteristic (e.g., an amplitude, pulse duration, pulse rate, duty cycle, electrode configuration or other stimulation parameter) to achieve or maintain the target or desired tongue movement. In an example, the HGN stimulator can use information from the accelerometer to determine whether the target or desired tongue movement can be achieved, and can communicate such information to a clinician, such as via remote monitoring for clinical intervention and titration of the therapy.

In an example, one or more of the sensor(s) 606 can be provided superior to, anterior to, or antero-superior to an upper airway obstruction to detect changes in airflow related to obstruction or relief of an obstruction. Sensor placement near or beyond an obstruction (e.g., in a direction away from the lungs) stands in contrast to prior methods that may include sensing information from the chest or thoracic trunk, near the lungs, and thus before the upper airway obstruction. The present inventors have recognized that some signals (sound, pressure, electrical, or otherwise) in the head or neck can be less subject to other (e.g., cardiac) signal interference and can be better correlated with airflow than, e.g., lung sounds. The present inventors have further recognized that some acoustic signals, such can be originate from or be measured at or near the trachea or nearby regions in the head or neck, can have higher amplitude and wider frequency spectrum or content than acoustic signals received at or adjacent to the lungs. The acoustic signals can be measured, for example using the accelerometer 712 or other transducer. The processor circuit 610 can receive the acoustic signals and, based on frequency and magnitude information from the signals, determine whether an airway of the patient is obstructed or partially obstructed. That is, the processor circuit 610 can use acoustic information to indicate an openness of the patient's airway. In an example, the processor circuit 610 can be configured to use the acoustic information to determine whether a patient is snoring, which can be an indication of partial airway obstruction.

In an example, a therapy can be modulated or updated based on patient posture information, such as head or neck posture information. Head or neck posture can be more influential on a patient's apnea hypopnea index (AHI) than, e.g., a chest or trunk posture. Further to modulating therapy based on posture or position, posture information can be used to modulate or help interpret information about tongue motion or information about an ECAP. For example, tongue motion information received from a head-supine position can be interpreted or processed differently than tongue motion information received from a head-lateral position. Similarly, different therapy parameters can be selected or indicated for use depending on a detected posture or position. That is, different electrostimulation parameters can be used to influence or achieve a particular tongue motion when the patient is in a head-supine position, while other electrostimulation parameters can be used to influence or achieve the same tongue motion when the patient is in a head-lateral position.

In an example, the HGN stimulator can be configured to provide

automatic neuromodulation by adjusting sets of therapy parameters to achieve target tongue movement, target ECAP response characteristics (e.g., amplitude or timing characteristics), or achieve unobstructed tracheal sounds (e.g., during sleep), such as depending on postural information of the head or neck. The therapy can optionally be determined in a titration sleep study, where the sensor information can be equated to a specific apnea/hypopnea relief signature in a particular patient, as a function of head or neck position or posture.

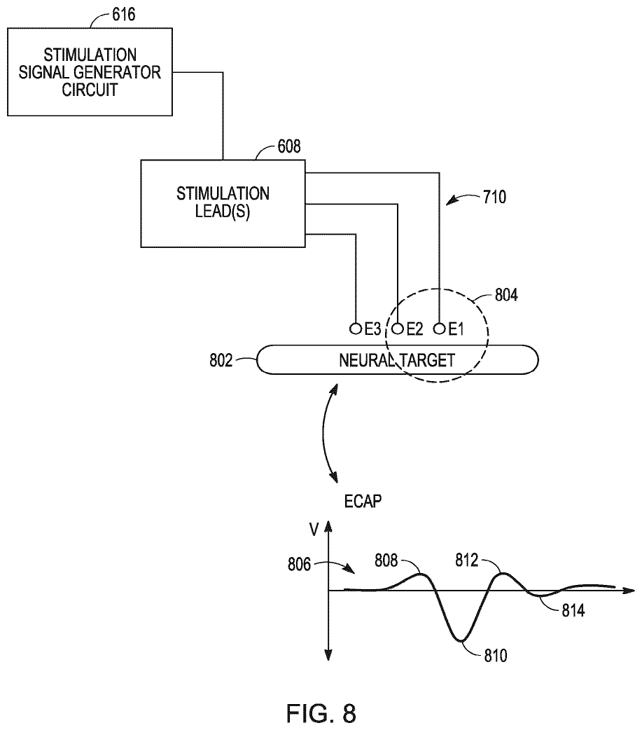

FIG. 8 illustrates generally an example of a portion of an implantable system, such as the implantable system 602, that is configured to provide a neurostimulation therapy and sense a patient response to the neurostimulation therapy. In an example, the stimulation signal generator circuit 616 is coupled to the stimulation lead(s) 608, and the stimulation lead(s) 608 include multiple, separately-addressable electrodes E1, E2, and E3. Additional or fewer electrodes can similarly be used. In an example, the electrodes illustrated in FIG. 8 correspond to the electrodes of the electrode array 710 from the example of FIG. 7. In some examples, at least one of the electrodes coupled to the stimulation signal generator circuit 616 is a conductive housing of an implantable device.

In the example of FIG. 8, the electrodes of the electrode array 710 are disposed at or adjacent to a neural target 802. The neural target 802 can include, for example, a branch of a hypoglossal nerve that, when stimulated using a therapy signal delivered by the electrodes, causes movement or displacement of a patient tongue, such as to treat a breathing disorder such as sleep apnea. In an example, the same or other electrodes of the electrode array 710, or of a different lead or array, can be used to sense an electrical response from the patient body. For example, the electrodes can receive an electrical signal at or near the neural target 802 that indicates whether or to what extent neural tissue was activated in response to a therapy signal. In an example, the electrodes can receive an electrical signal about an evoked compound action potential, or ECAP signal 806, indicative of the neural tissue response.

An ECAP is sensed after delivery of a neurostimulation signal, such as after a specified delay duration, and includes various signal peaks and valleys. The timing and magnitude (or amplitude) characteristics of the ECAP signal 806 can indicate particular neural target or neural fiber activations. Since the ECAP signal 806 is sensed using a pair of spaced-apart electrodes, the ECAP signal 806 includes information about a response from a tissue area or volume that includes a number of neural fibers. In the example of FIG. 8, the ECAP signal 806 includes a first positive peak 808 (sometimes referred to as P1), a first negative peak 810 (sometimes referred to as N1), a second positive peak 812 (P2), a second negative peak 814 (N2), and so on. In an example, information about the timing and magnitude of the different peaks and valleys can be used (e.g., by the processor circuit 610) to determine an effectiveness of the therapy signal in activating a particular neural pathway. For example, a peak ECAP characteristic (e.g., a timing or magnitude characteristic of a positive or negative peak of an ECAP response) measured from a cranial nerve may be used as a surrogate that indicates a magnitude of a muscle response, and in turn indicates tongue position or an openness or obstruction of an airway.

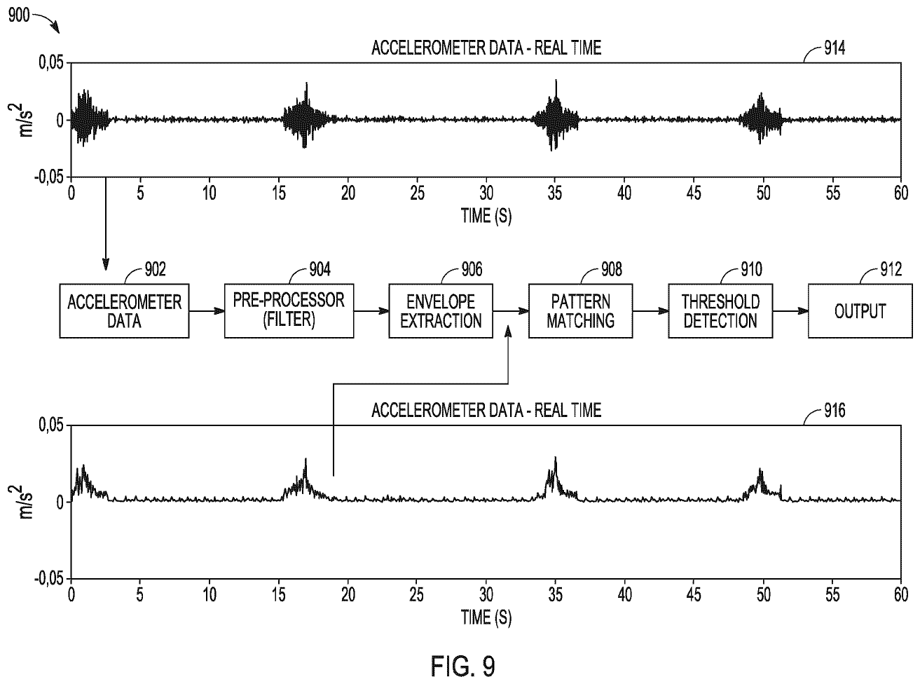

FIG. 9 illustrates generally an example of a physiologic parameter detection algorithm 900 that can include or use an input signal that comprises sensor data from one or more sensor(s) 606. For example, the algorithm can use accelerometer information from the accelerometer 712. In the example of FIG. 9, accelerometer data 902 can include an accelerometer signal 914. The accelerometer signal 914 can include a digital representation of acceleration data corresponding to one or multiple axes. In an example where the accelerometer 712 is implanted in a mandibular, cervical, or thoracic region of a patient, the accelerometer data 902 can include information about various physiologic parameters, including movement associated with respiration, tracheal sounds (breathing), tongue movement, heart rate, patient orientation, and more. The accelerometer data 902 can include data sampled from one or more accelerometers 712 at one or more respective sample rates and data resolutions.

The accelerometer data 902 can be processed using various filters and analyses. For example, the accelerometer data 902 can be used to determine information about patient respiration by pre-processing 904 or filtering the accelerometer data 902. In an example, pre-processing 904 can include band-pass filtering the data with a pass-band of approximately 30 Hz to 60 Hz, or in other pass-bands that include, or are likely to include, information about patient respiration activity. In an example, pre-processing 904 includes filtering the data with other low-pass or high-pass filters to identify respiration-related characteristics. For example, pre-processing 904 can include low-pass filtering with a cutoff frequency of one or more Hertz. Signals around or below 1 Hz can be associated with respiration-related movements.

In an example, pre-processing 904 can include or use a high-pass filter, such as an nth-order (e.g., 4th-order) Butterworth filter, with Fc=30 Hz (or other corner frequency). The filter can help eliminate out-of-band noise, such as may be dominated by 1/f noise. Together with a sampling rate of, e.g., 100 Hz, the filtered result is a signal bandwidth of about 30 Hz to 50 Hz. Removing any DC components in this manner, however, can correspondingly remove information about patient position or orientation. Accordingly, the system can be configured to periodically or intermittently reconfigure its filtering and processing to acquire the orientation information, as further described below.

In an example, the pre-processed accelerometer data 902 can be further processed through envelope extraction 906 such as to determine an envelope characteristic of the accelerometer signal. In an example, envelope extraction 906 can include rectification and low-pass filtering, such as with a cutoff frequency of, e.g., 1 Hz. FIG. 9 illustrates generally an example of an accelerometer envelope signal 916 for the accelerometer data accelerometer signal 914. In an example, envelope extraction 906 can include or use a Hilbert transform on the results from the pre-processing 904. In another example, envelope extraction 906 can be performed using rectification (e.g., through an absolute value function) and applying a low-pass filter. In an example, the envelope extraction 906 can include or use a smoothing filter, such as can include a 4th order IIR low-pass Butterworth filter with Fc=1 Hz.

In an example, the envelope signal can be further processed using pattern matching 908. The pattern matching 908 can include or use a matched filter with an order of N (e.g., 256). The N filter coefficients can be determined using empirical respiration data collected and averaged to create a template for convolution or matched filtering. In an example, pattern matching 908 can be configured to pattern “match” the filtered accelerometer signal 914 to that of an expected template, and an output of the matched filter can indicate a “match” with a large value output.

In an example, the matched filter comprises a convolution of the template and the sensed signal, where the template includes information about the signal of interest. The output of the matched filter “spikes” when it detects that the template signal exists in the sensed signal.

Additionally or alternatively to pattern matching, a peak-picking technique can be used. In an example, this technique includes tracking a sensed signal over a specified period of time and calculating a mean and standard deviation of the signal values. In an example, the specified period can include several seconds of signal value data, such as 3 seconds. When a current signal value exceeds the historical mean plus standard deviation, then a peak is detected.

The pattern-matched result or peak-detected result can be processed using a threshold detector 910. A threshold (e.g., a patient-specific threshold) can be set and used to identify particular events, or identify timing characteristics of particular events, in the processed or filtered accelerometer signal 914. For example, an output 912 can include a binary indication of whether the filtered accelerometer signal 914 meets or exceeds a specified threshold condition. One or multiple different threshold values can be specified for detecting different peaks or different peak levels in the filtered accelerometer signal 914. In an example, the interface 626 of the external system 620 can be configured to set or update the threshold values.

In an example, the filter parameter or coefficients used for the pattern matching 908 can be updated or adjusted. A patient device can be programmed after implantation (e.g., days or weeks after implantation) and a sleep study can be performed. During this study, the clinician can optimize device settings by monitoring the efficacy of the implant while the patient sleeps. During the study, various polysomnography instruments can be used to monitor the patient to help validate the measurements of the implanted device. For example, pulse, orientation, respiration rate, etc., can all be tracked and can be compared to the values reported by the implant. Differences between physiologic parameter values measured by the implanted device and by other instruments can indicate a need to update or change parameters or coefficients of the template used for the pattern matching 908 in the physiologic parameter detection algorithm 900.

Additionally or alternatively to performing the pattern matching and threshold detection described in FIG. 9, the system can be configured to analyze timing characteristics of various features of the raw or filtered accelerometer signal to identify physiologic status information about the patient. In an example, accelerometer signal features that correspond to physiological events can be identified, and timing information about the signal features can be used to determine the patient's respiration cycle, posture, activity level, or heart rate, among other parameters.

For respiration cycle determination, for example, the system can be configured to identify peaks and troughs within the raw or filtered accelerometer signal that correspond to the inhalation and exhalation phases of the patient's breathing. The time intervals between successive peaks or troughs can be measured to determine the respiration rate. In some examples, the duration and amplitude of these features can be measured and used to provide information about a depth or regularity of breathing.

In an example, the system can analyze static and dynamic components of the accelerometer signal to determine the patient's posture and activity level, respectively. The static component, which reflects the orientation of the patient relative to gravity, can be characterized by the signal's baseline level over time. Changes in this baseline can indicate transitions between different postures, such as sitting, standing, or lying down. The dynamic component, which includes information about patient motion, can be characterized by the frequency and amplitude of transient signal features. The occurrence and pattern of these transients can reveal the patient's activity type or activity level, and can be used to distinguish between periods of rest and physical activity, or to distinguish restful sleep from non-restful sleep.

In an example, the system can be configured to determine a patient heart rate by identifying periodic features of the accelerometer signal that correspond to the mechanical activity of the heart. The system can detect the repetitive patterns associated with the heartbeats and measure the intervals between them to calculate the heart rate. The regularity and variability of these intervals can also provide insights into the patient's cardiac function and health status.

The present inventors have recognized that the physiologic parameter detection algorithm 900 can optionally be performed in parallel, using multiple instances or copies of the same accelerometer signal 914 (or intermediately processed portions of the accelerometer signal 914) to identify different physiologic parameters such as patient orientation or posture, activity level, respiration, heart rate, etc. However, performing such operations in parallel can drain processing and power resources on the implantable system 602. The present inventors have further recognized that some physiologic parameters may not change significantly or meaningfully over short durations; for example, pulse rate and orientation may be unlikely to materially change over the course of a single respiration cycle. Accordingly, the present inventors have recognized that the implantable system 602 can be designed and tuned to track particular physiologic parameters in time-multiplexed, clinically-relevant intervals. In this manner, fewer processing resources or a single group of processing resources can be used, thereby reducing or minimizing the drain on processing and power resources of the implantable system 602.

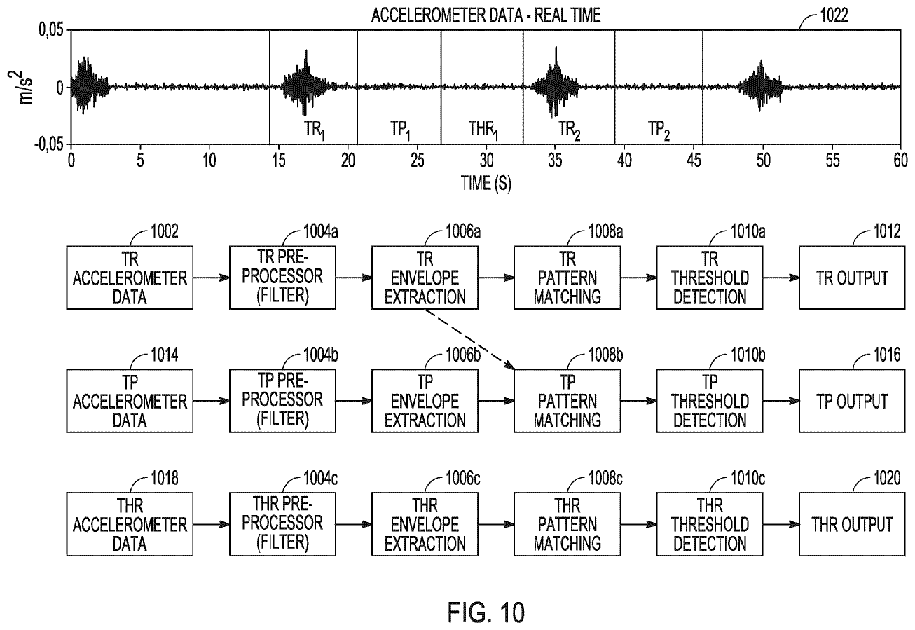

FIG. 10 illustrates generally an example of a time-divided accelerometer signal 1022, such as can comprise the accelerometer signal 914 with various time divisions or intervals. The time divisions can indicate which portions of the accelerometer signal 914 are used for different analyses. That is, information from the different intervals can be separately processed to yield respective outputs indicative of respective different physiologic parameters.

The time-divided accelerometer signal 1022 can include respiration cycle intervals designated TRᵢ. A first respiration cycle interval, TR₁, can indicate a particular respiration cycle including one inhalation event and one corresponding exhalation event for a patient. A second respiration cycle interval, TR₂, can indicate a subsequent respiration cycle for the patient. A respiration refractory period, or dwell time, between the onsets of the first and second respiration cycle intervals can be fixed or variable. For example, an onset of a respiration cycle interval can be determined based on expiration of one or more other intervening intervals, based on historical patient information or a patient-specific template, or based on other physiologic information from the patient.

In an example, the time-divided accelerometer signal 1022 can include posture sensing intervals designated TPᵢ, heart rate sensing intervals designated THRᵢ, and so on for different physiologic parameters or status information. The example of FIG. 10 indicates that a first posture sensing interval, TP₁, can include an interval following or adjacent to an end of the first respiration cycle interval, and a first heart rate sensing interval, THR₁, can follow the first posture sensing interval. Posture or patient orientation information can be derived from the portions of the time-divided accelerometer signal 1022 corresponding to the TPᵢ intervals, and heart rate (or other pulse characteristic information) can be derived from the information in the time-divided accelerometer signal 1022 corresponding to the THR₁ intervals.

FIG. 10 illustrates generally various algorithm or processing blocks that can be applied or used together to process the accelerometer data to yield different physiologic parameter information. For example, a first group of processing blocks 1004a through 1010a can be used to determine respiration information by applying uniquely tuned filters to process the TR accelerometer data 1002 corresponding to one or more of the respiration sensing intervals of the time-divided accelerometer signal 1022. That is, parameters of one or more of the TR pre-processing 1004a, TR envelope extraction 1006a, TR pattern matching 1008a, and/or TR threshold detector 1010a can be selected to optimize respiration sensing or respiratory cycle sensing. The first group of processing blocks can yield a TR output 1012 with information about patient respiration, such as corresponding to one or multiple respiratory cycles. In an example, the TR output 1012 includes information about whether an apnea event was detected during one or more of the respiration sensing intervals. In an example, the TR output 1012 includes information about inspiration phase timing. A delay in detecting an expected inspiration phase, or an absence of an expected inspiration phase, can indicate an apnea event or probable apnea event. Other information about patient respiration can be provided, such as respiration phase duration information, apnea duration information, or an indication of severity of the apnea event.

A second group of processing blocks 1004b through 1010b can be used to determine posture or orientation information by applying uniquely tuned filters to process the TP accelerometer data 1014 corresponding to one or more of the posture sensing intervals of the time-divided accelerometer signal 1022. For example, parameters of one or more of the TP pre-processing 1004b, TP envelope extraction 1006b, TP pattern matching 1008b, and/or TP threshold detector 1010b can be selected to optimize posture or orientation sensing. The second group of processing blocks can yield a TP output 1016 with information about patient posture.

In an example, determining orientation can include determining the angle for gravity and comparing it to previous values over time to determine changes in orientation. In an example, orientation can be calculated using the angle of inclination data for each axis measured by a multiple-axis accelerometer. In an example, determining orientation can be performed prior to or separately from any high-pass or band pass filtering of the accelerometer signal.

In an example, one or more processor blocks may be common to different filtering or processing chains for different physiologic parameters. For example, each of heart rate and posture sensing algorithms can include or use the same pre-processing filtering at 1004a and 1004b. In this example, the TP envelope extraction 1006b can be configured to receive a processing result of the TR pre-processing 1004a to further save processing resources. Other processing or filtering blocks can be similarly combined or consolidated to help optimize performance and reduce usage of compute resources.

A third group of processing blocks 1004c through 1010c can be used to determine heart rate or other pulse characteristic information by applying uniquely tuned filters to process the THR accelerometer data 1018 corresponding to one or more of the heart rate sensing intervals of the time-divided accelerometer signal 1022. For example, parameters of one or more of the THR pre-processing 1004c, THR envelope extraction 1006c, THR pattern matching 1008c, and/or THR threshold detector 1010c can be selected to optimize heart rate or pulse characteristic sensing. The third group of processing blocks can yield a THR output 1020 with information about a patient heart rate or other pulse characteristic.

In an example, the TR accelerometer data 1002, the TP accelerometer data 1014, and/or the THR accelerometer data 1018 can include or represent data received from the same or different accelerometer 712 and can include data sampled at the same or respective different rates. For example, a sample rate for the TP accelerometer data 1014 can be lower than a sample rate used to collected the TR accelerometer data 1002.

In an example, the various pre-processing blocks can be differently tuned for the different physiologic parameters to be detected. For example, the TR pre-processing 1004a can include or use a filter to isolate respiration information, such as at or below 1 Hz for tissue motion information, or in a range of about 30 to 60 Hz for acoustic information related to respiration. The TP pre-processing 1004b can include or use a low-pass filter with a cutoff around 1 Hz. The THR pre-processing 1004c can include or use a band-pass filter to isolate pulse information, such as in the range of 20 to 200 Hz. Other ranges can similarly be used.

In an example, the envelope extraction blocks, such as can include or use rectification and low-pass filtering, can be differently tuned for the different physiologic parameters to be detected. For example, the TR envelope extraction 1006a and the TP envelope extraction 1006b can use a low-pass cutoff frequency of about 1 Hz, while the THR envelope extraction 1006c can use a cutoff frequency of about 200 Hz.

The pattern matching blocks can generally be configured to apply convolution operations with respective matched filters. At TR pattern matching 1008a, the filter coefficients can be tuned for respiration sensing, at TP pattern matching 1008b, the filter coefficients can be tuned for orientation sensing, and at THR pattern matching 1008c, the filter coefficients can be tuned for pulse rate or pulse characteristic detection. In some examples, pattern matching may be omitted or can include a pass-through filter (e.g., for posture sensing when a clean signal is available).

The threshold detection blocks can generally be configured with programmable thresholds for peak detection in the various processed and filtered signals. For example, the TR threshold detector 1010a can be programmed with specific thresholds for respiration cycle feature detection, the TP threshold detector 1010b can be programmed with specific thresholds for orientation detection, and the THR threshold detector 1010c can be programmed with specific thresholds for pulse detection.

The time-multiplexed physiologic information sensing enables the system to gather a wide array of physiological data from a single source, such as an implanted accelerometer, thereby minimizing the need for multiple sensors and reducing the system's power consumption. Time-multiplexed sensing can include dividing the accelerometer signal into distinct time intervals, or durations, each of which can be processed to yield information about respective types of physiological status. For instance, certain intervals can be used to determine respiration information, while others can be used to determine a patient's posture, heart rate, or other characteristics. The division into different intervals can be dynamically managed based on the patient's current state and the specific data requirements at any given time. Furthermore, not all physiologic parameters may need to be sensed at the same frequency.

By employing time-multiplexed sensing, the system can adaptively prioritize and switch between different sensing modalities. This adaptability is helpful for monitoring conditions that exhibit variability over time, such as sleep disorders, where the patient's physiological state can change throughout the night. Furthermore, the systems and methods discussed herein enable more efficient use of the implanted device's processing and battery resources, extending the device's operational lifespan and reducing the frequency of charging or other maintenance.

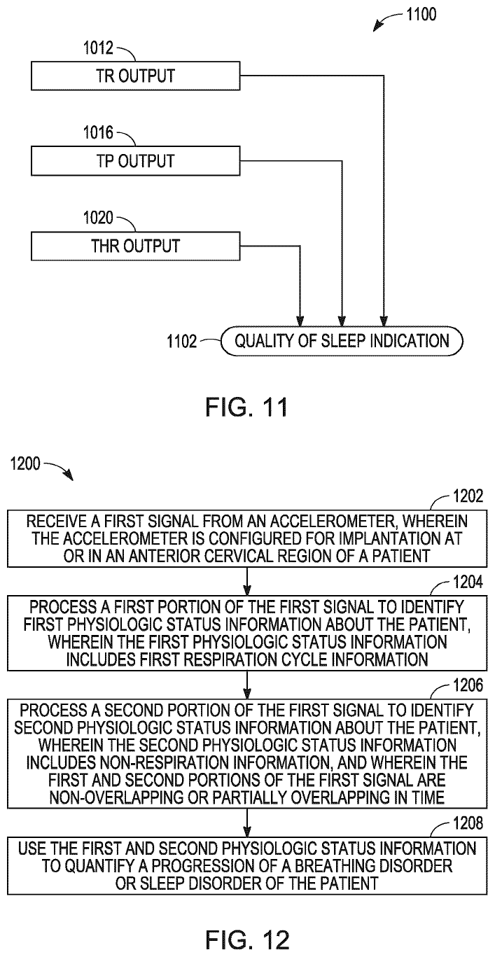

FIG. 11 illustrates generally an example of a method 1100 for evaluating a quality of sleep for a patient. For example, the implantable system 602 and/or the external system 620 can be configured to provide a quality of sleep indication 1102 for a patient based on information from the accelerometer signal 914. In an example, the method 1100 can include receiving, at a processor circuit, the TR output 1012 (with information about the respiration of the patient), the TP output 1016 (with information about the orientation or posture of the patient), and the THR output 1020 (with information about the heart rate or pulse of the patient). In an example, the method 1100 can include receiving, at the processor circuit, information about one or more other patient characteristics, such as including activity level information about the patient. The processor circuit can analyze the various received information together and provide the quality of sleep indication 1102.

The method 1100 for evaluating a quality of sleep for a patient leverages the data that can be extracted or determined from the accelerometer signal 914 to provide a more thorough understanding of the patient's sleep health. The integration of respiration data (e.g., from the TR output 1012), posture, orientation, or activity level data (e.g., from the TP output 1016), and/or heart rate or pulse data or other activity level data (e.g., from the THR output 1020) allows for a multi-dimensional analysis of sleep quality. This approach recognizes that quality sleep is not solely about the absence of sleep disorders such as obstructive sleep apnea but also involves stable and appropriate physiological conditions throughout the sleep cycle. For instance, frequent changes in posture or orientation, or an elevated heart rate or other increased activity level, may indicate restless sleep or underlying stress, even in the absence of breathing interruptions.

In an example, the method 1100 can including tracking changes in sleep quality over time, thereby providing further insights for both patients and healthcare providers. By comparing the quality of sleep indication 1102 across different nights, or different periods during a single night, the system can identify patterns or trends that may be related to lifestyle factors, medication changes, or the progression of sleep-related disorders. This analysis can help inform personalized treatment plans and lifestyle recommendations aimed at improving sleep health.

FIG. 12 illustrates generally an example of a routine 1200 for quantifying a progression of a patient disorder, such as a breathing disorder or sleep disorder. In operation 1202, routine 1200 receives a first signal from an accelerometer, wherein the accelerometer is configured for implantation at or in an anterior cervical region of a patient. In operation 1204, routine 1200 processes a first portion of the first signal to identify first physiologic status information about the patient, wherein the first physiologic status information includes first respiration cycle information. In operation 1206, routine 1200 processes a second portion of the first signal to identify second physiologic status information about the patient, wherein the second physiologic status information includes non-respiration information, and wherein the first and second portions of the first signal are non-overlapping or partially overlapping in time. In operation 1208, routine 1200 uses the first and second physiologic status information to quantify a progression of a breathing disorder or sleep disorder of the patient.

At operation 1202, the routine 1200 begins with receiving a first signal from an accelerometer. In some examples, the accelerometer comprises a portion of an implantable system that is designed or configured for implantation at or in the anterior cervical region of a patient. This placement of the accelerometer allows for the collection of data that is highly relevant to the patient's physiological status, particularly in relation to sleep and breathing disorders. The signal received from the accelerometer can include a wide variety of physiologic status-indicating information, including information about subtle movements that may indicate breathing patterns and more pronounced movements that could signify changes in posture or activity level during sleep or at other times.

At operation 1204, the routine 1200 includes processing a first portion of the received accelerometer signal to identify first physiologic status information about the patient, for example including first respiration cycle information. Operation 1204 can include analyzing the accelerometer signal to identify patterns or signal components that correspond to the patient's breathing. This can include detecting movements or acoustic signals associated with inhalation and exhalation.

At operation 1206, the routine 1200 includes processing a second portion of the accelerometer signal to identify second physiologic status information about the patient, for example including non-respiration information. The non-respiration information can include, for example, information about the patient's posture or orientation during sleep, heart rate, or other pulse characteristics. In an example, the first and second portions of the accelerometer signal are non-overlapping or are partially overlapping in time.

At operation 1208, the routine 1200 includes using the first and second physiologic status information to provide a patient health status indication. In an example, the patient health status indication includes quantitative information about a progression of a breathing disorder or sleep disorder of the patient. In an example, operation 1208 includes synthesizing the data collected and processed in the previous steps to assess the severity or progression of the patient's condition. By comparing the physiologic status information against known patterns or indicators of sleep and breathing disorders, the routine 1200 can provide insights into the patient's health, potentially identifying issues that can benefit from intervention.

In other words, the patient health status indication can be determined using a combination of respiration cycle information and non-respiration cycle information. The respiration cycle information includes metrics such as respiration rate, depth of breathing, and the presence of any irregularities such as apnea or hypopnea events. Non-respiration cycle information encompasses data related to the patient's posture, activity level, or heart rate, among other information, each of which contributes to an understanding of the patient's health, particularly during sleep.

The patient health status indication can be used to inform clinical decisions, such as the adjustment of neuromodulation therapy parameters to optimize treatment efficacy. For example, if the system detects a pattern of apnea events, then the system can automatically update or adjust neurostimulation parameters, such as can include increasing stimulation intensity or duration to prevent airway obstruction. Similarly, if the patient's activity level data indicates poor sleep quality, then the system can make adjustments to the timing or pattern of stimulation to help promote better sleep.

In an example, the patient health status indication can be used for long- term health monitoring and management. By tracking changes in the patient's physiological data over time, the system can help identify trends or emerging issues that may warrant medical attention. This proactive approach to patient care enables early intervention and personalized treatment strategies, ultimately improving patient outcomes.

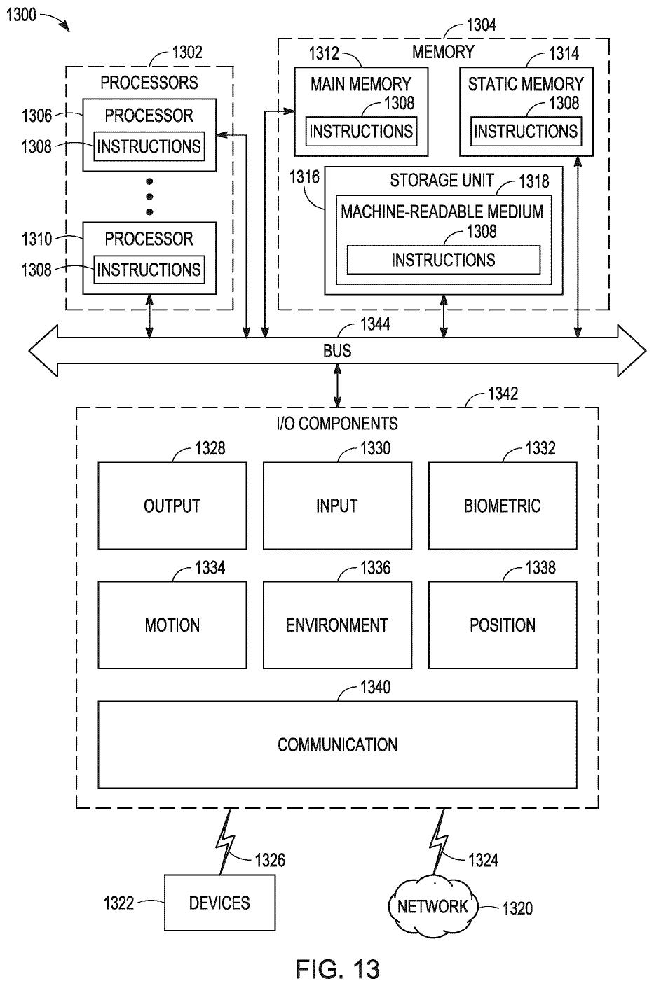

FIG. 13 is a diagrammatic representation of a machine 1300 within which instructions 1308 (e.g., software, a program, an application, an applet, an app, or other executable code) for causing the machine 1300 to perform any one or more of the methodologies discussed herein may be executed. The machine 1300 can optionally comprise the implantable system 602, the external system 620, or components or portions thereof, or components or devices that can be coupled to at least one of the implantable system 602 and the external system 620.

In an example, the instructions 1308 may cause the machine 1300 to execute any one or more of the methods, controls, therapy algorithms, signal processing algorithms, signal generation routines, or other processes described herein. The instructions 1308 transform the general, non-programmed machine 1300 into a particular machine 1300 programmed to carry out the described and illustrated functions in the manner described. The machine 1300 may operate as a standalone device or may be coupled (e.g., networked) to other machines. In a networked deployment, the machine 1300 may operate in the capacity of a server machine or a client machine in a server-client network environment, or as a peer machine in a peer-to-peer (or distributed) network environment. The machine 1300 can comprise, but is not limited to, various systems or devices that can communicate with the implantable system 602 or the external system 620, such as can include a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1308, sequentially or otherwise, that specify actions to be taken by the machine 1300. Further, while only a single machine 1300 is illustrated, the term “machine” shall also be taken to include a collection of machines that individually or jointly execute the instructions 1308 to perform any one or more of the methodologies discussed herein.

The machine 1300 may include processors 1302, memory 1304, and I/O components 1342, which may be configured to communicate with each other via a bus 1344. In an example embodiment, the processors 1302 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio- Frequency Integrated Circuit (RFIC), another processor, or any suitable combination thereof) may include, for example, a processor 1306 and a processor 1310 that execute the instructions 1308. The term “processor” is intended to optionally include multi-core processors that may comprise two or more independent processors (sometimes referred to as “cores”) that may execute instructions contemporaneously. Although FIG. 13 shows multiple processors 1302, the machine 1300 may include a single processor with a single core, a single processor with multiple cores (e.g., a multi-core processor), multiple processors with a single core, multiple processors with multiples cores, or any combination thereof.

The memory 1304 includes a main memory 1312, a static memory 1314, and a storage unit 1316, both accessible to the processors 1302 via the bus 1344. The main memory 1304, the static memory 1314, and storage unit 1316 store the instructions 1308 embodying any one or more of the methodologies or functions described herein. The instructions 1308 may also reside, completely or partially, within the main memory 1312, within the static memory 1314, within a machine-readable medium 1318 within the storage unit 1316, within at least one of the processors 1302 (e.g., within the processor's cache memory), or any suitable combination thereof, during execution thereof by the machine 1300.

The I/O components 1342 may include a variety of components to receive input, provide output, produce output, transmit information, exchange information, capture measurements, and so on. The specific I/O components 1342 that are included in a particular machine will depend on the type of machine. For example, portable machines such as device programmers or mobile phones may include a touch input device or other such input mechanisms, while a headless server machine will likely not include such a touch input device. It will be appreciated that the I/O components 1342 may include other components that are not shown in FIG. 13. In various example embodiments, the I/O components 1342 may include output components 1328 and input components 1330. The output components 1328 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1330 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), physiologic sensor components, and the like.

In further example embodiments, the I/O components 1342 may include biometric components 1332, motion components 1334, environmental components 1336, or position components 1338, among others. For example, the biometric components 1332 can include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 1334 can include an acceleration sensor (e.g., an accelerometer), gravitation sensor components, rotation sensor components (e.g., a gyroscope), or similar. The environmental components 1336 can include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1338 can include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1342 further include communication components 1340 operable to couple the machine 1300 to a network 1320 or other devices 1322 via a coupling 1324 and a coupling 1326, respectively. For example, the communication components 1340 may include a network interface component or another suitable device to interface with the network 1320. In further examples, the communication components 1340 may include wired communication components, wireless communication components, cellular communication components, Near Field Communication (NFC) components, Bluetooth components, or Wi-Fi components, among others. The devices 1322 may be another machine or any of a wide variety of peripheral devices such as can include other implantable or external devices.

The various memories (e.g., memory 1304, main memory 1312, static memory 1314, and/or memory of the processors 1302) and/or storage unit 1316 can store one or more sets of instructions and data structures (e.g., software) embodying or used by any one or more of the methodologies or functions described herein. These instructions (e.g., the instructions 1308), when executed by processors 1302, cause various operations to implement the disclosed embodiments, including various neuromodulation or neurostimulation therapies or functions supportive thereof.

To better illustrate the systems and methods described herein, such as can be used to optimize an electrostimulation therapy to treat a sleep disorder or breathing disorder for a patient, a non-limiting set of Example embodiments are set forth below as numerically identified Examples.

Example 1 is a method for sensing physiologic status information using an implanted sensor, the method comprising receiving a first signal from an accelerometer, wherein the accelerometer is configured for implantation at or in an anterior cervical region of a patient, and processing a first portion of the first signal to identify first physiologic status information about the patient, wherein the first physiologic status information includes respiration cycle information (e.g., inhalation and/or exhalation information, including timing information about an onset, duration, or termination of an inhalation and/or exhalation phase), and processing a second portion of the first signal from the accelerometer to identify second physiologic status information about the patient. The second physiologic status information can include non-respiration information about the patient. Example 1 can further include providing a patient health status indication using the first and second physiologic status information.

In Example 2, the subject matter of Example 1 optionally includes the first and second portions of the first signal are non-overlapping in time.

In Example 3, the subject matter of any one or more of Examples 1-2 optionally includes the first and second portions of the first signal are at least partially overlapping in time.

In Example 4, the subject matter of any one or more of Examples 1-3 optionally includes processing the first portion of the first signal by: applying a first filter to isolate respiration information from noise and yield a first filtered signal; determining an envelope characteristic of the first filtered signal to yield an envelope signal; applying a pattern matching algorithm to the envelope signal to yield a first respiration signal; and providing the respiration cycle information based on a comparison of the first respiration signal to one or more specified threshold values.

In Example 5, the subject matter of Example 4 optionally includes determining parameters of the first filter and/or parameters of the pattern matching algorithm using information about the patient acquired from a sleep study.

In Example 6, the subject matter of any one or more of Examples 4-5 optionally includes processing the second portion of the first signal to identify at least one of information about an orientation of the patient, information about a heart rate of the patient, information about an activity level of the patient, or information about a pulse characteristic of the patient.

In Example 7, the subject matter of Example 6 optionally includes processing the second portion of the first signal, including applying a second filter to isolate the second physiologic status information from noise and yield a second filtered signal; determining an envelope characteristic of the second filtered signal; and providing the second physiologic status information based on a comparison of the envelope characteristic to one or more specified threshold values.

In Example 8, the subject matter of Example 7 optionally includes processing a third portion of the first signal to identify subsequent respiration cycle information about the patient, wherein the first and subsequent respiration cycle information correspond to different respiration cycles for the patient.

In Example 9, the subject matter of Example 8 optionally includes processing the first and third portions of the first signal to identify the subsequent respiration cycle information about the patient including applying respective pattern matching filters.

In Example 10, the subject matter of any one or more of Examples 1-9 optionally includes providing the patient health status indication including quantifying a progression or state of a breathing disorder or a sleep disorder of the patient.

In Example 11, the subject matter of Example 10 optionally includes quantifying the progression of the breathing disorder or sleep disorder of the patient including identifying a number of apnea events for the patient for a specified unit time.

In Example 12, the subject matter of Example 11 optionally includes quantifying the progression of the breathing disorder or sleep disorder for the patient including determining a respiratory rate, a heart rate, and/or an orientation or posture of the patient.

In Example 13, the subject matter of any one or more of Examples 11-12 optionally includes the second portion of the first signal corresponds to a refractory period for respiration for the patient.

In Example 14, the subject matter of any one or more of Examples 1-13 optionally includes receiving the first signal from the accelerometer including receiving information about acceleration of the accelerometer along multiple axes.

In Example 15, the subject matter of any one or more of Examples 1-14 optionally includes processing a third portion of the first signal to identify acoustic information about inhalation or exhalation that corresponds to the respiration cycle information from the first physiologic status information, and validating the respiration cycle information from the first physiologic status information based on the identified acoustic information from the third portion of the first signal.

In Example 16, the subject matter of any one or more of Examples 1-15 optionally includes determining, from the respiration cycle information, at least one of an inspiration timing characteristic or an exhalation timing characteristic for a particular respiratory cycle of the patient, and processing a third portion of the first signal to identify third physiologic status information about the patient, wherein the third physiologic status information includes respiration cycle validation information. Example 16 can include validating the inspiration timing characteristic or the exhalation timing characteristic using the respiration cycle validation information.

In Example 17, the subject matter of Example 16 optionally includes the first and third portions of the first signal are substantially overlapping in time.

In Example 18, the subject matter of any one or more of Examples 16-17 optionally includes the first physiologic status information including lower-frequency information (e.g., infrasound information) about displacement or motion of the accelerometer during the particular respiratory cycle, and the third physiologic status information including higher-frequency information (e.g., acoustic information) about tracheal sounds during the particular respiratory cycle.

In Example 19, the subject matter of any one or more of Examples 1-18 optionally includes receiving the first signal from the accelerometer including receiving the first portion of the first signal at a first sampling rate and receiving the second portion of the first signal at a different second sampling rate.

Example 20 is a system comprising: a first housing configured for implantation in an anterior cervical region of a patient; a first electrode lead coupled to the first housing and configured to be disposed in a submandibular region, wherein at least one electrode on the first electrode lead is configured to be disposed at or near a first branch of a hypoglossal nerve of the patient; an accelerometer configured to provide a sensor signal that includes, information about (1) a patient response to a first electrostimulation therapy that is provided to the patient using the at least one electrode, wherein the first electrostimulation therapy is configured to treat a sleep disorder or breathing disorder of the patient, (2) a respiration of the patient, and (3) a physiologic status characteristic of the patient, the physiologic status characteristic including information about one or more of an orientation, activity level, heart rate, or pulse characteristic of the patient; and a processor circuit configured to provide an indication of a breathing disorder status for the patient based on the patient response, the respiration, and the physiologic status characteristic of the patient.

In Example 21, the subject matter of Example 20 optionally includes the processor circuit configured to: determine the patient response to the first electrostimulation therapy using a first sampled portion of the sensor signal; determine the respiration of the patient using a second sampled portion of the sensor signal; and determine the physiologic status characteristic of the patient using a third sampled portion of the sensor signal to determine the physiologic status characteristic of the patient. In Example 21, the first, second, and third sampled portions of the sensor signal optionally correspond to non-overlapping time intervals.

In Example 22, the subject matter of any one or more of Examples 20-21 optionally includes the processor circuit configured to adjust the first electrostimulation therapy based on the indication of the breathing disorder status.

In Example 23, the subject matter of any one or more of Examples 20-22 optionally includes respective portions of the sensor signal corresponding to the information about the respiration of the patient and the physiologic status characteristic of the patient.

Example 24 is a system for sensing physiologic status information using an implanted sensor, the system comprising: an accelerometer configured for implantation at or in an anterior cervical region of a patient and configured to provide a first sensor signal, and a processor circuit configured to: process a first portion of the first sensor signal to identify first physiologic status information about the patient, wherein the first physiologic status information includes first respiration cycle information; process a second portion of the first sensor signal to identify second physiologic status information about the patient, wherein the second physiologic status information includes non-respiration information. In Example 24, the first and second portions of the first signal can be non-overlapping or can be at least partially overlapping in time. Example 24 can further include a therapy delivery circuit configured to provide a neurostimulation therapy to the patient based on the first and second physiologic status information.

In Example 25, the subject matter of Example 24 optionally includes the therapy delivery circuit configured to provide a quantitative indication of a progression of a breathing disorder or sleep disorder of the patient.

In Example 26, the subject matter of any one or more of Examples 24-25 optionally includes the first portion of the first sensor signal comprising information about timing characteristics of one or more inspiration and exhalation phases of the patient.

In Example 27, the subject matter of Example 26 optionally includes the second portion of the first sensor signal comprises information about activity level, posture, and pulse characteristics of the patient.

In Example 28, the subject matter of Example 27 optionally includes the respective non-overlapping portions of the first sensor signal correspond to the information about the activity level, posture, and pulse characteristics of the patient.

In Example 29, the subject matter of Example 28 optionally includes the pulse characteristics comprise information about a heart rate of the patient.

In Example 30, the subject matter of any one or more of Examples 24-29 optionally includes the processor circuit configured to: apply a first signal filter to the first sensor signal to isolate respiration information from noise and yield a first filtered signal; determine an envelope characteristic of the first filtered signal to yield an envelope signal; apply a pattern matching algorithm to the envelope signal to yield a first respiration signal; and provide the first respiration cycle information based on a comparison of the first respiration signal to one or more specified threshold values.

In Example 31, the subject matter of Example 30 optionally includes or uses parameters of the first signal filter and parameters of the pattern matching algorithm that are based on information about the patient acquired from or during a sleep study of the patient.

In Example 32, the subject matter of any one or more of Examples 30-31 optionally includes the processor circuit configured to process the second portion of the first sensor signal to identify at least one of an activity level, posture, and pulse characteristic (e.g., heart rate, pulse strength, heart rate variability, etc.) of the patient.

In Example 33, the subject matter of Example 32 optionally includes the processor circuit configured to: apply a second filter to isolate the second physiologic status information from noise and yield a second filtered signal; determine an envelope characteristic of the second filtered signal; and provide information about the activity level, posture, and pulse characteristics of the patient based on a comparison of the envelope characteristic to one or more specified threshold values.

Example 34 is at least one machine-readable medium including instructions that, when executed by processing circuitry, cause the processing circuitry to perform operations to implement of any of Examples 1-33.

Example 35 is an apparatus comprising means to implement of any of Examples 1-33.

Each of these non-limiting examples can stand on its own, or can be combined in various permutations or combinations with one or more of the other examples.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part, such as using the implantable system 602, the external system 620, the machine 1300, or using the other systems, devices, or components discussed herein. Some examples can include a computer-readable medium or machine-readable medium encoded with instructions operable to configure an electronic device to perform methods, such as neuromodulation therapy control methods, as described in the above examples, such as to treat one or more diseases or disorders. In an example, the instructions can include instructions to receive sensor data from one or more physiologic sensors and, based on the sensor data, titrate a therapy. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1-33. (canceled)

34. A method for evaluating sleep quality of a patient using an implanted sensor, the method comprising:
receiving a sensor signal from an accelerometer, the accelerometer configured for implantation at or in an anterior cervical region of a patient;
processing a first portion of the sensor signal to identify first physiologic status information about the patient, the first physiologic status information including respiration cycle information of the patient;
processing a second portion of the sensor signal to identify second physiologic status information about the patient, the second physiologic status information including non-respiration information of the patient;
determining a quality of sleep indication based on the first physiologic status information and the second physiologic status information; and
providing the quality of sleep indication via an external system.

35. The method of claim 34, further comprising:
processing a third portion of the sensor signal to identify subsequent first physiologic status information about the patient, the subsequent first physiologic status information including subsequent respiration cycle information;
processing a fourth portion of the sensor signal to identify subsequent second physiologic status information about the patient, the subsequent second physiologic status information including subsequent non-respiration information;
determining an updated quality of sleep indication based on the subsequent first physiologic status information and the subsequent second physiologic status information; and
providing the updated quality of sleep indication via the external system.

36. The method of claim 35, wherein determining the updated quality of sleep indication is further based on the first physiologic status information and the second physiologic status information.

37. The method of claim 35, further comprising comparing the quality of sleep indication and the updated quality of sleep indication.

38. The method of claim 34, wherein the first and second portions of the sensor signal are non-overlapping in time.

39. The method of claim 34, wherein the first and second portions of the sensor signal are at least partially overlapping in time.

40. The method of claim 34, further comprising processing a third portion of the sensor signal to identify third physiologic status information about the patient, the third physiologic status information including non-respiratory information of the patient different than the second physiologic status information; wherein
determining the quality of sleep indication is further based on the third physiologic status information about the patient.

41. The method of claim 40, wherein:
the second physiologic status information comprises posture, orientation, or activity level information; and
the third physiologic status information comprises heart rate or pulse data.

42. The method of claim 34, wherein processing the second portion of the sensor signal includes identifying at least information about an orientation of the patient, information about a heart rate of the patient, information about an activity level of the patient, or information about a pulse characteristic of the patient.

43. The method of claim 34, wherein the second physiologic status information comprises posture or orientation information, and determining the quality of sleep indication comprises analyzing a frequency of changes in posture or orientation.

44. The method of claim 34, comprising identifying a number of apnea events for the patient for a specified unit time.

45. A system for sensing physiologic status information using an implanted sensor, the system comprising:
an accelerometer configured for implantation at or in an anterior cervical region of a patient and configured to provide a sensor signal;
a processor circuit configured to:
process a first portion of the sensor signal to identify first physiologic status information about the patient, the first physiologic status information including first respiration cycle information;
process a second portion of the sensor signal to identify second physiologic status information about the patient, the second physiologic status information including non-respiration information; and
determine a quality of sleep indication based on the first physiologic status information and the second physiologic status information; and
output the quality of sleep indication.

46. The system of claim 45, wherein the processor circuit is further configured to:
process a third portion of the sensor signal to identify subsequent first physiologic status information about the patient, the subsequent first physiologic status information including subsequent respiration cycle information;
process a fourth portion of the sensor signal to identify subsequent second physiologic status information about the patient, the subsequent second physiologic status information including subsequent non-respiration information; wherein
the processor circuit is configured to determine updated quality of sleep indication based on the subsequent first physiologic status information and the subsequent second physiologic status information.

47. The system of claim 46, wherein the processor circuit is further configured to compare the quality of sleep indication and the updated quality of sleep indication.

48. The system of claim 45, wherein the processor circuit is further configured to:
process a third portion of the sensor signal to identify third physiologic status information about the patient, the third physiologic status information including non-respiratory information of the patient different than the second physiologic status information; wherein
the processor circuit is configured to determine the quality of sleep indication further based on the third physiologic status information about the patient.

49. The system of claim 48, wherein:
the second physiologic status information comprises information about orientation or posture of the patient; and
the third physiologic status information comprises information about a heart rate or pulse of the patient.

50. The system of claim 45, wherein:
the processor circuit is configured to identify a number of apnea events for the patient for a specified unit time.

51. The system of claim 45, further comprising a therapy delivery circuit configured to provide a neurostimulation therapy to the patient based on the first and second physiologic status information.

52. The system of claim 45, comprising an external system comprising the processor circuit and an interface, and wherein the processor circuit is configured to output the quality of sleep indication via the interface.

53. The system of claim 52, comprising an implantable system comprising the accelerometer and further comprising a wearable device in communication with at least the external system or the implantable system, the wearable device comprising at least a biometric component, a motion component, or a position component.

54. The system of claim 45, comprising an implantable system comprising the accelerometer and the processor circuit and an external system comprising an interface, and wherein the processor circuit of the implantable system is configured to output the quality of sleep indication to the external system for display via the interface.

55. The system of claim 54, further comprising a wearable device in communication with at least the external system or the implantable system, the wearable device comprising at least a biometric component, a motion component, or a position component.
